(19) United States
(12) Patent Application Publication
(10) Pub. No.: US 2026/0070918 A1
(43) Pub. Date: Mar. 12, 2026

(54) HETEROBICYCLIC COMPOUNDS AS EP4 RECEPTOR ANTAGONISTS

(71) Applicants: Shenzhen Ionova Life Science Co., Ltd., Shenzhen (CN); Foshan Ionova Biotherapeutics Co., Inc., Foshan (CN); Guangdong Touchstone Translational Research Institute Co., Ltd., Foshan (CN)

(72) Inventors: Jianming Bao, Shenzhen (CN); Weiguo Quan, Shenzhen (CN); Ying-Duo Gao, Shenzhen (CN); Yongkui Sun, Shenzhen (CN)

(73) Assignees: Shenzhen Ionova Life Science Co., Ltd., Shenzhen (CN); Foshan Ionova Biotherapeutics Co., Inc., Foshan (CN); Guangdong Touchstone Translational Research Institute Co., Ltd., Foshan (CN)

(21) Appl. No.: 19/106,046

(22) PCT Filed: Aug. 23, 2023
(86) PCT No.: PCT/CN2023/114414

§ 371 Date: Feb. 24, 2025

Related U.S. Application Data
(60) Provisional application No. 63/425,555, filed on Nov. 15, 2022.

(30) Foreign Application Priority Data
Aug. 24, 2022 (WO) PCT/CN2022/114402

(51) Int. Cl. C07D 495/04; A61K 31/407; A61K 39/395; A61P 19/02; A61P 35/00

(52) U.S. Cl. CPC C07D 495/04; A61K 31/407; A61K 39/39558; A61P 19/02; A61P 35/00

(57) ABSTRACT

The invention is directed to a series of novel heterobicyclic amide derivatives as EP4 receptor antagonists which are useful for treating diseases or conditions mediated by the action of PGE2 at EP4 receptors, such as pain, an inflammatory disease and cancer. Also within the scope of this invention are pharmaceutical compositions containing such compounds and methods of use of these compounds for treating a subject suffering from a condition mediated by the action of PGE2 at EP4 receptors.

### CROSS-REFERENCE TO RELATED APPLICATIONS

This application claims priority to international application number PCT/CN2022/114402 (filed on Aug. 24, 2022) and US provisional application number U.S. 63/425,555 (filed on Nov. 15, 2022), the contents of both of which are incorporated herein by reference in their entirety.

### BACKGROUND OF THE INVENTION

This invention relates to heterocyclic amide derivatives, or their pharmaceutically acceptable salts, pharmaceutical acceptable prodrugs, pharmaceutical compositions made therefrom, and their medical uses in mammals including humans. The compounds of this invention have activity as prostaglandin E2 (PGE2) receptor antagonists, which are useful in treatment or alleviation of pain, inflammation, and cancer.

Prostaglandins are a group of physiologically active lipid compounds having diverse hormone-like effects in the body. They're interesting because they can have different effects depending on the receptors where they attach. Studies have showed that prostaglandins are important mediators of pain, fever and other symptoms associated with inflammation. Of particular interest is prostaglandin E2 (PGE2), which is the predominant eicosanoid associated with inflammation. Moreover, a highly expressed PGE2 in tumor tissues suppresses antitumor immunity in the tumor microenvironment (TME) and causes tumor immune evasion leading to disease progression (see, Front Immunol. 2020; 11: 324). PGE2 has also been shown to promote colorectal cancer stem cell (CSC) expansion and metastasis in mice (Wang et al., Gastroenterology, 2015, 1-12). The activity of PGE2 is largely mediated by its binding to a panel of G-protein-coupled receptors (GPCRs), namely E-type prostanoid (EP) receptors EP1, EP2, EP3 and EP4. Among them, the EP4 receptor is currently emerging as most versatile and promising among PGE2 receptors. In animal studies, selectively inhibition of the PGE2/EP4 receptor signaling by antagonists reduces tumor growth (Terada et al. Cancer Res. 2010, 70, 1606-1615) and tumor metastasis (Yang et al. Cancer Res. 2006, 66, 9665-9672).

Up to now, EP4 receptor antagonists of different structural classes have been described. It is the object of the present invention to provide a series of novel heterobicyclic compounds as EP4 receptor antagonists and methods for treating diseases or conditions mediated by the action of PGE2 at EP4 receptors, including pain, inflammation and cancer, and pharmaceutical compositions thereof.

### BRIEF SUMMARY OF THE INVENTION

The invention is directed to a series of novel heterobicyclic amide derivatives as EP4 receptor antagonists, which are useful for treating diseases or conditions mediated by the action of PGE2 at EP4 receptors, such as pain, inflammation, and cancer. Pharmaceutical compositions and methods of use are also included.

In one aspect, the present invention provides a heterobicyclic compound or pharmaceutically acceptable salt thereof. The compound has a structure of Formula I as shown below:

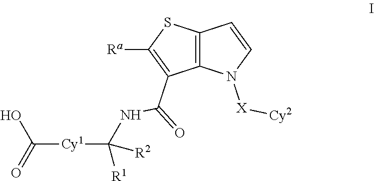

wherein:
R¹ and R² are each independently hydrogen, C₁₋₆ alkyl, C₁₋₆ cycloalkyl, C₁₋₆ halo-cycloalkyl, and C₁₋₆ haloalkyl; or, R¹ and R², together with the carbon atom to which they are both attached to, form a 3- to 6-membered carbocyclic ring which is optionally substituted with one to three R^{a} groups and optionally contains one or two ring-forming heteroatom(s) each independently being S, O, or NR^{b}, wherein each R^{b} is independently hydrogen, C₁₋₆ alkyl, C₁₋₆ cycloalkyl, C₁₋₆ halo-cycloalkyl, and C₁₋₆ haloalkyl, aryl, heteroaryl, —C(O)—C₁₋₆ alkyl, —C(O)-aryl, —S(O)₂-alkyl or —S(O)₂-aryl;
X is absent, ═CH—, —CR¹R²—, or —C(O)—;
Cy¹ is C₁₋₆ alkylene, C₁₋₆ alkenylene, C₁₋₆ alkynylene, cycloalkylene, arylene, heteroarylene, heterocyclylene, or bridged bicyclic cycloalkylene, and is optionally substituted;
Cy² is cycloalkyl, aryl, heteroaryl, heterocyclyl, and each of cycloalkyl, aryl, heteroaryl, heterocyclyl is optionally substituted with one to three substitution groups each of which is independently halo, alkyl, or haloalkyl groups; and
each R^{a} independently is halo, alkyl, haloalkyl, hydroxyalkyl, or alkoxy, and when R^{a} is alkyl, Cy¹ is bridged bicyclic cycloalkylene.

In some embodiments, X is CH₂.

In some embodiments, Cy² is aryl optionally substituted with one haloalkyl.

In some embodiments, halo is —F or —Cl.

In some embodiments, Cy¹ is arylene or bridged bicyclic cycloalkylene.

In some embodiments, R¹ and R² are each independently hydrogen or C₁₋₆ alkyl; or, R¹ and R², together with the carbon atom to which they are both attached, form a 3- to 6-membered carbocyclic ring.

In some embodiments, R^{a} is —F, —Cl, —CF₃, hydroxyalkyl, alkoxyl or —CH₃, with the provision that when R^{a} is —CH₃, Cy¹ is C₅-C₁₀ bridged bicyclic cycloalkylene.

In some other embodiments, the compound is Formula II:

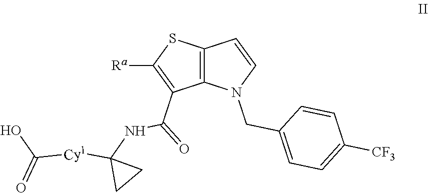

in which, Cy¹ is arylene or bridged bicyclic cycloalkylene; and R^{a} is halo, alkyl, haloalkyl, hydroxyalkyl, or alkoxy, provided that when R^{a} is alkyl, Cy¹ is bridged bicyclic cycloalkylene.

In still other embodiments, Cy¹ is phenylene or C₅-C₁₀ bridged bicyclic cycloalkylene. Examples of the C₅-C₁₀ bridged bicyclic cycloalkylene include, but are not limited to

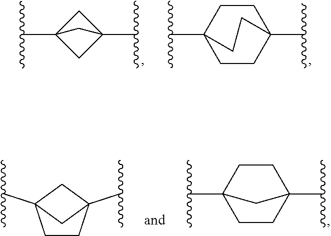

Examples of the compounds of this invention include:

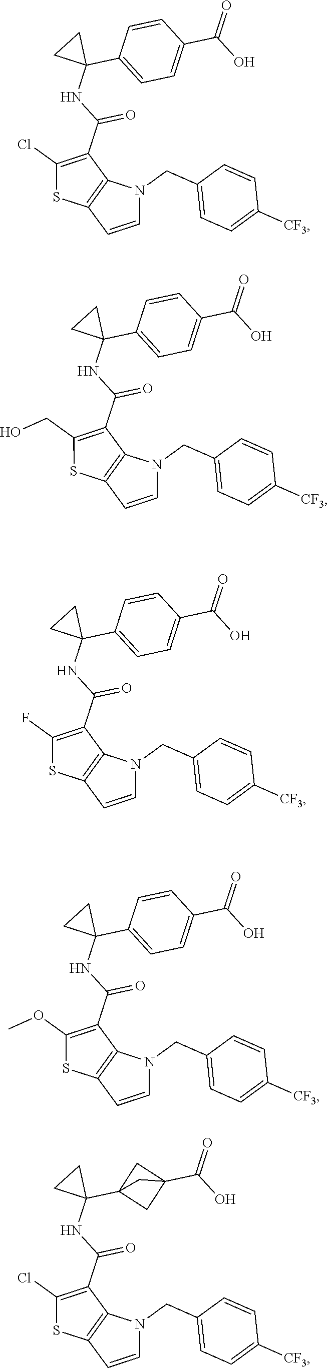 [SMILES: O=C(O)c1ccc(C2(NC(=O)c3c(Cl)sc4ccn(Cc5ccc(C(F)(F)F)cc5)c34)CC2)cc1 ; O=C(O)c1ccc(C2(NC(=O)c3c(CO)sc4ccn(Cc5ccc(C(F)(F)F)cc5)c34)CC2)cc1 ; O=C(O)c1ccc(C2(NC(=O)c3c(F)sc4ccn(Cc5ccc(C(F)(F)F)cc5)c34)CC2)cc1 ; COc1sc2ccn(Cc3ccc(C(F)(F)F)cc3)c2c1C(=O)NC1(c2ccc(C(=O)O)cc2)CC1 ; O=C(NC1(C23CC(C(=O)O)(C2)C3)CC1)c1c(Cl)sc2ccn(Cc3ccc(C(F)(F)F)cc3)c12]

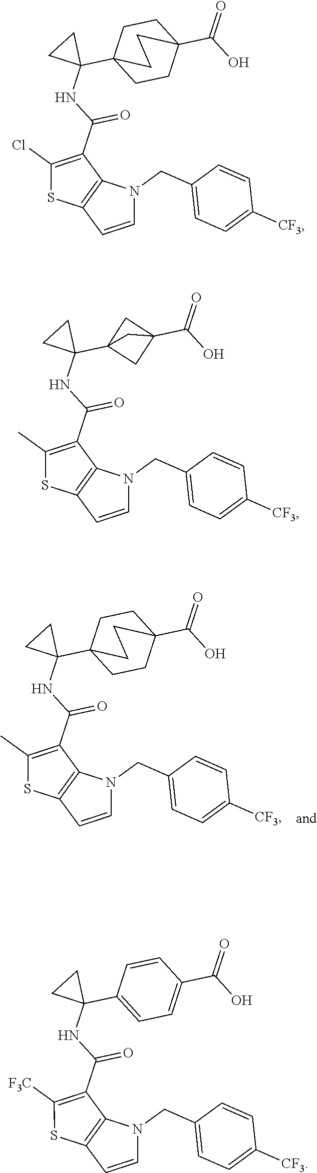 [SMILES: O=C(NC1(C23CCC(C(=O)O)(CC2)CC3)CC1)c1c(Cl)sc2ccn(Cc3ccc(C(F)(F)F)cc3)c12 ; Cc1sc2ccn(Cc3ccc(C(F)(F)F)cc3)c2c1C(=O)NC1(C23CC(C(=O)O)(C2)C3)CC1 ; Cc1sc2ccn(Cc3ccc(C(F)(F)F)cc3)c2c1C(=O)NC1(C23CCC(C(=O)O)(CC2)CC3)CC1 ; O=C(O)c1ccc(C2(NC(=O)c3c(C(F)(F)F)sc4ccn(Cc5ccc(C(F)(F)F)cc5)c34)CC2)cc1]

Another aspect of this invention provides pharmaceutical compositions each of which includes a compound as described or a pharmaceutically acceptable salt thereof, and a pharmaceutically or physiologically acceptable carrier or excipient.

In some embodiments, such pharmaceutical composition includes another therapeutic agent which can be an antibody to cytotoxic t-lymphocyte antigen 4 (anti-CTLA4), an antibody to programmed death ligand 1 (anti-PDL1), an antibody to programmed cell death protein 1 (anti-PD1), an indoleamine-2,3-dioxygenase (IDO) inhibitor, a tryptophan-2,3-dioxygenase (TDO) inhibitor or antimetabolite.

In some embodiments, such pharmaceutical compositions are used in combination with a radiation therapy agent.

Yet still another aspect of this invention provides a method for treating a subject suffering from a condition mediated by the action of PGE2 at EP4 receptors, comprising administering to the subject in need thereof an effective amount of a compound or a pharmaceutical composition as described above.

In some embodiments, the condition is an inflammatory disease or cancer.

Examples of the inflammatory disease include, but are not limited to, arthritis, acne vulgaris, asthma, autoimmune diseases, autoinflammatory diseases, Celiac disease, chronic prostatitis, colitis, diverticulitis, glomerulonephritis, hidradenitis suppurativa, hypersensitivities, inflammatory bowel diseases, interstitial cystitis, Mast Cell Activation Syndrome, macrocytosis, otitis, pelvic inflammatory disease, reperfusion injury, rheumatic fever, rheumatoid arthritis, rhinitis, sarcoidosis, and or vasculitis.

Examples of the cancer include, but are not limited to, breast cancer, endometrial cancer, cervix cancer, ovary cancer, lung cancer, head and neck cancer, brain cancer, thyroid cancer, esophagus cancer, stomach cancer, colon & rectal cancer, liver cancer, pancreatic cancer, skin cancer, kidney cancer, bladder cancer, prostate cancer, testis cancer, bone cancer, Lymphoma, and blood cancer.

Also within the scope of this invention is use of a compound described above for manufacture of a medicament for treating a subject suffering from a condition mediated by the action of PGE2 at EP4 receptors.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications, and equivalents, which may be included within the spirit and scope of the invention as defined by the claims. Furthermore, in the detailed description of the present invention, numerous specific details are set forth to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

### Definitions

As used herein, the term “or” is meant to include both “and” and “or”. In other words, the term “or” may also be replaced with “and/or”.

As used herein, the singular forms “a”, “an” and “the” are intended to include the plural forms as well, unless the context clearly indicates otherwise.

As used herein, the term “subject” or “patient” is used interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

As used herein, the term “unsaturated bond” refers to a double or triple bond.

As used herein, the term “unsaturated” or “partially unsaturated” refers to a moiety that includes at least one double or triple bond.

As used herein, the term “saturated” refers to a moiety that does not contain a double or triple bond, i.e., the moiety only contains single bonds.

As used herein, the term “alkyl” by itself or as part of another substituent refers to a straight (i.e., unbranched) or branched hydrocarbon chain radical consisting of carbon and hydrogen atoms, containing no unsaturation, having the stated number of carbon atoms (e.g., C₁-C₁₀ or C₁₋₁₀ alkyl). Whenever it appears herein, a numerical range such as “1 to 10” refers to each integer in the given range, e.g., “1 to 10 carbon atoms” means that the alkyl group can consist of 1 carbon atom, 2 carbon atoms, 3 carbon atoms, 4 carbon atoms, etc., up to and including 10 carbon atoms, although the present definition also covers the occurrence of the term “alkyl” where no numerical range is designated. Representative saturated linear or straight alkyl includes, but not limited to, -methyl, -ethyl, -n-propyl, -n-butyl, -n-pentyl, and -n-hexyl; while saturated branched alkyl includes, but not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more substituents.

As used herein, the term “halo” or “halogen” refers to fluorine (fluoro, —F), chlorine (chloro, —Cl), bromine (bromo, —Br), or iodine (iodo, —I). “Haloalkyl” refers to alkyl as defined above in which one or more of the hydrogen atoms have been replaced with a halogen independently selected from fluoro, chloro, bromo and iodo. “Fluoroalkyl” means alkyl as defined above wherein one or more hydrogen atoms have been replaced by fluoro atoms.

As used herein, the term “alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl, propenyl, and the like.

As used herein, the term “alkynyl” by itself or as part of another substituent refers to carbon chains which contain at least one carbon-carbon triple bond, and which may be linear or branched or combinations thereof. Examples of alkynyl include ethynyl, propargyl, 3-methyl-1-pentynyl, 2-heptynyl and the like.

As used herein, the term “cycloalkyl” by itself or as part of another substituent refers to a cyclic version of an “alkyl” group. A cycloalkyl group may include zero bridgehead carbon atoms or two or more bridgehead carbon atoms. Thus, a cycloalkyl may be monocyclic, bicyclic, or polycyclic, depending upon the number of bridgehead and bridging carbon atoms. Cycloalkyl groups that include zero bridgehead carbon atoms are referred to herein as “monocyclic cycloalkyls” or “unbridged cycloalkyls.” Cycloalkyls that include at least two bridgehead carbon atoms and at least one bridging carbon atom are referred to herein as “bridged cycloalkyls.” Bridged cycloalkyls that include two bridgehead carbon atoms are referred to herein as “bicyclic bridged cycloalkyls” or “bridged bicyclic cycloalkyls”. Bridged cycloalkyls that include more than two bridgehead carbon atoms are referred to herein as “polycyclic bridged cycloalkyls” or “bridged polycyclic cycloalkyls”. A “lower” unbridged cycloalkyl contains from 3 to 8 carbon atoms. A “lower” bridged cycloalkyl contains from 5 to 16 carbon atoms.

The term “Cycloalkenyl” as used herein is a non-aromatic carbon-based ring composed of at least three carbon atoms and containing at least one carbon-carbon double bond, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like.

The term “heterocycloalkenyl” is a type of cycloalkenyl group as defined above, wherein at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted.

The term “heterocyclyl,” as used herein, refers to a group derived from a monocyclic, bicyclic, or polycyclic compound comprising at least one nonaromatic ring comprising one or more, preferably one to three, heteroatoms independently selected from nitrogen, oxygen, and sulfur. The heterocyclyl groups of the present disclosure can be attached to the parent molecular moiety through a carbon atom or a heteroatom in the group.

As used herein, the term “hydroxyl” or “hydroxy” refers to the group —OH.

The above-defined groups may include prefixes and/or suffixes that are commonly used in the art to create additional well-recognized substituent groups. As examples, the term “alkyloxy” or “alkoxy” refers to a group of the formula —OR, “alkylamine” refers to a group of the formula —NHR and “dialkylamine” refers to a group of the formula —NRR, where each R is independently an alkyl. As another example, “haloalkoxy” or “haloalkyloxy” refers to a group of the formula —OR′, where R′ is a haloalkyl.

The term “hydroxyalkyl” by itself or as part of another substituent refers to an alkyl group in which one or more of the hydrogen atoms are replaced with a hydroxyl substituent. Thus, the term “hydroxyalkyl” is meant to include, e.g., monohydroxyalkyls, dihydroxyalkyls, trihydroxyalkyls, etc.

As used herein, the term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl).

As used herein, the term “heteroaryl” refers to a radical of a monocyclic or polycyclic (e.g., bicyclic or tricyclic) 4n+2 aromatic ring system (e.g., having 6, 10, or 14π electrons shared in a cyclic array) having ring carbon atoms and one or more ring heteroatoms provided in the aromatic ring system, wherein each heteroatom is independently selected from nitrogen, oxygen, and sulfur. In heteroaryl groups that contain one or more nitrogen atoms, the point of attachment can be a carbon or nitrogen atom, as valency permits. Heteroaryl polycyclic ring systems can include one or more heteroatoms in one or both rings.

The term “alkylene” by itself or as part of another substituent refers to a bidentate moiety obtained by removing two hydrogen atoms, either both from the same carbon atom, or one from each of two different carbon atoms, of a hydrocarbon compound, which may be aliphatic or alicyclic, and which may be saturated, partially unsaturated, or fully unsaturated. Thus, the term “alkylene” includes the sub-classes alkenylene, alkynylene, cycloalkylene, etc. In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, refers to an alkylene group having from 1 to 4 carbon atoms.

Examples of linear saturated C₁₋₈ alkylene groups include, but are not limited to, —(CH₂)ₙ— where n is an integer from 1 to 8, for example, —CH₂—, —CH₂CH₂CH₂—, and —CH₂CH₂CH₂CH₂—.

Examples of branched saturated C₁₋₇ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₇alkylene groups include, but is not limited to, —CH═CH—, —CH═CH—CH₂—, —CH═CH—CH₂—CH₂—, —CH═CH—CH₂CH₂CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₇alkylene groups include, but is not limited to, —C(CH₃)═CH—, —C(CH₃)═CH—CH₂—, and —CH═CH—CH(CH₃)—.

Examples of alicyclic saturated C₁₋₇alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₇alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

The term “arylene,” as used herein, refers to a bidentate moiety obtained by removing two hydrogen atoms, one from each of two different aromatic ring atoms of an aromatic compound, which moiety has from 3 to 20 ring atoms (unless otherwise specified). Preferably, each ring has from 5 to 7 ring atoms.

As used herein, the term “absent” that defines a variable, such as X, means that the variable is not present, and thus the two groups that connected through the variable are directly connected to each other. For example, in —N—X-Cy₂ when X is absent, Cy₂ and N are directly connected to each other.

The term “optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with the alkyl group.

### Isomeric Forms

The present invention provides novel compounds of Formula I, or a pharmaceutically acceptable salts therefore, as EP4 receptor antagonists, which are useful in treating PGE2 mediated diseases or conditions.

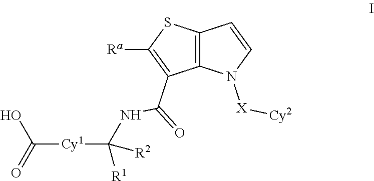

It will be appreciated that certain compounds of Formula I (or salts, prodrugs, or conjugates) may exist in, and be isolated in, isomeric forms, including tautomeric forms, cis- or trans-isomers, as well as optically active, racemic, or diastereomeric forms. It is to be understood that the present invention encompasses a compound of Formula I in any of the tautomeric forms or as a mixture thereof; or as a mixture of diastereomers, as well as in the form of an individual diastereomers, and that the present invention encompasses a compound of Formula I as a mixture of enantiomers, as well as in the form of an individual enantiomer, any of which mixtures or form possesses antagonistic properties against EP4 receptor. It is well known in the art how to prepare or isolate particular forms and how to determine antagonistic properties against EP4 receptor by standard tests including those described herein below.

In addition, a compound of Formula I (or salt, prodrug or conjugate thereof) may exhibit polymorphism or may form a solvate with water or an organic solvent. The present invention also encompasses any such polymorphic form, any solvate or any mixture thereof.

Compounds of Formula I contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Alternatively, any enantiomer of a compound of Formula I may be obtained by stereospecific synthesis using optically pure starting materials or reagents of known configuration.

### Salts

The term “pharmaceutically acceptable salts” refers to salts prepared from pharmaceutically acceptable non-toxic bases or acids including inorganic or organic bases and inorganic or organic acids, which are, within the scope of sound medical judgment, suitable for use in contact with the tissues of patients without excessive toxicity, irritation, allergic response, or other problem or complication commensurate with a reasonable benefit/risk ratio and are effective for their intended use. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethyl-aminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methyl-glucamine, morpholine, piperazine, piperidine, polyamine resins, procaine, purines, theobromine, triethylamine, trimethylamine, tripropylamine, tromethamine, and the like.

When a compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Prodrug

As used herein, the term “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrugs may also have improved solubility in pharmaceutical compositions over the parent drug. An example, without limitation, of a prodrug would be a compound of Formula I, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug, again without intending to limit the scope of the term, might be one in which a short peptide is bonded to an acid group which is converted to the active moiety inside the cell.

The invention also encompasses acceptable forms of prodrugs of compound of Formula I, which is formed in a conventional manner with a functional group of the compound such as with an amino, hydroxy, or carboxy group.

### Utilities

Compounds of the present invention are antagonists of the EP4 receptor and are therefore expected to be useful in treating EP4 receptor mediated diseases.

In one aspect, the invention encompasses a method of treating a human or animal subject suffering from a condition which is mediated by the action of PGE2 at EP4 receptors, which comprises administering to the subject an effective amount of a compound of Formula I.

In another aspect, this invention encompasses use of a compound of Formula I for the manufacture of a medicament for the treatment of a disease or condition that is mediated by the action of PGE2 at EP4 receptors.

As used herein, the term “treating a prostaglandin E2 (PGE2) mediated disease or condition” or “treatment of a disease or condition that is mediated by the action of PGE2 at EP4 receptors” means treating or preventing any chronic disease or condition that is advantageously treated or prevented by selective EP4 antagonists. The term includes the relief of pain, fever and inflammation of a variety of conditions including rheumatic fever, symptoms associated with influenza or other viral infections, common cold, low back pain, neck pain, dysmenorrhea, headache, migraine, toothache, sprains and strains, myositis, neuralgia, synovitis, arthritis, including rheumatoid arthritis, degenerative joint diseases (osteoarthritis), gout, ankylosing spondylitis, bursitis, burns, injuries, and pain and inflammation following surgical procedures. In addition, such a compound may inhibit cellular neoplastic transformations and metastatic tumor growth and hence can be used in the treatment and/or prevention of cancer.

Examples of cancers include, but are not limited to, breast cancers, cancers which can be related to Li-Fraumeni syndrome, for example, childhood sarcomas, leukemias and brain cancers, cancers which can be related to Lynch syndrome, for example, colon cancers, bile duct cancers, brain cancers, endometrial cancers, kidney cancers, ovarian cancers, pancreatic cancers, small intestinal cancers, stomach cancers and ureter cancers, lung cancers, melanomas, prostate cancers, retinoblastomas, thyroid cancers and uterine cancers. Moreover, cancer can be the result of acquired mutations, for example, mutations resulting from diet, environment and/or lifestyle, or somatic mutations. Examples of such cancers may include, but are not limited to, adrenal cancer, adrenal cortex cancer, bladder cancer, brain cancer, primary brain cancer, glioma, glioblastoma, breast cancer, cervical cancer, colon cancer (non-limiting examples include colorectal carcinomas such as colon adenocarcinoma and colon adenoma), endometrial cancer, epidermal cancer, esophageal cancer, gall bladder cancer, genitourinary cancer, head or neck cancer, kidney cancer, liver cancer, lung cancer (non-limiting examples include adenocarcinoma, small cell lung cancer and non-small cell lung cancer), lymphomas (non-limiting examples include B-cell lymphoma, T-cell lymphoma, Hodgkin's lymphoma, non-Hodgkin's lymphoma), melanoma, malignant melanoma, malignant carcinoid carcinoma, malignant pancreatic insulinoma, myeloma, multiple myeloma, ovarian cancer, pancreatic cancer (such as exocrine pancreatic carcinoma), prostate cancer, renal cell cancer, skin cancer, such as, in addition to others previously mentioned, squamous cell carcinoma, stomach cancer, testicular cancer, thyroid cancer, thyroid follicular cancer, Wilms' tumor, choriocarcinoma, mycosis fungoides, malignant hypercalcemia, cervical hyperplasia, leukemia, acute lymphocytic leukemia, chronic lymphocytic leukemia, hairy cell lymphoma, Burkett's lymphoma, acute myelogenous leukemia, chronic myelogenous leukemia, myelodysplastic syndrome, promyelocytic leukemia, chronic granulocytic leukemia, acute granulocytic leukemia, fibrosarcoma, rhabdomyosarcoma, astrocytoma, neuroblastoma, rhabdomyosarcoma, schwannoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, seminoma, teratocarcinoma, osteosarcoma, xeroderma pigmentosum, keratoacanthoma and retinoblastoma.

As used herein, the terms “treat”, “treating” or “treatment” and the like refer to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters, including the results of a physical examination, neurological examination, and/or psychiatric evaluations.

An “effective amount” or “treatment-effective amount” refers to an amount that is effective for treating a prostaglandin E2 mediated disease or condition or a cancer as noted through clinical testing and evaluation, patient observation, and/or the like. An “effective amount” can further designate an amount that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition. An “effective amount” can further refer to a therapeutically effective amount.

The compounds of Formula I can also be used in combination with radiation and/or one or more therapeutic agents selected from chemotherapeutic agents, antibodies to cytotoxic t-lymphocyte antigen 4 (anti-CTLA4), antibodies to programmed death ligand 1 (anti-PD-L1), antibodies to programmed cell death protein 1 (anti-PD1), indoleamine-2,3-dioxygenase (IDO) inhibitors, tryptophan-2,3-dioxygenase (TDO) inhibitors, and antimetabolites. Example of these antibodies include, but not limited to, MDX-010 (ipilimumab, Bristol-Myers Squibb), CP-675,206 (tremelimumab, Pfizer), MPDL3280A (Roche), MDX-1106 (nivolumab, Bristol-Myers Squibb), lambrolizumab (Merck), and pembrolizumab (KEYTRUDA®, Merck).

Examples of chemotherapeutic agents include, but not limited to, an aromatase inhibitor; an antiestrogen, an anti-androgen (especially in the case of prostate cancer) or a gonadorelin agonist; a topoisomerase I inhibitor or a topoisomerase II inhibitor; a microtubule active agent, an alkylating agent, an antineoplastic antimetabolite or a platin compound; a compound targeting/decreasing a protein or lipid kinase activity or a protein or lipid phosphatase activity, a further anti-angiogenic compound or a compound which induces cell differentiation processes; a bradykinin I receptor or an angiotensin II antagonist; a cyclooxygenase inhibitor, a bisphosphonate, a rapamycin derivative such as everolimus, a heparanase inhibitor (prevents heparan sulphate degradation), e.g. PI-88, a biological response modifier, preferably lymphokine or interferon, e.g. interferon, an ubiquitination inhibitor, or an inhibitor which blocks anti-apoptotic pathways; an inhibitor of Ras oncogenic isoforms, e. g. H-Ras, K-Ras or N-Ras, or a farnesyl transferase inhibitor, e.g. L-744, 832 or DK8G557; a telomerase inhibitor, e.g., telomestatin; a protease inhibitor, a matrix metalloproteinase inhibitor, a methionine aminopeptidase inhibitor, e.g. bengamide or a derivative thereof, or a proteasome inhibitor, e.g. PS-341; a histone deacetylase inhibitor, e.g. Vorinostat, MG0103 or MS275; and kinase inhibitors.

The pharmaceutical compositions of the present invention comprise a compound of Formula I as an active ingredient or a pharmaceutically acceptable salt, thereof, and may also contain a pharmaceutically acceptable carrier and optionally other therapeutic ingredients.

The EP4 antagonist, antibody and/or anti-metabolite can be administered to subjects by any suitable route, including orally (inclusive of administration via the oral cavity and further including administration via an orogastric feeding tube), intraperitoneally, parenterally, by inhalation spray, topically (i.e., both skin and mucosal surfaces, including airway surfaces), transdermally, rectally, nasally (including a nasogastric feeding tube), sublingually, buccally, vaginally or via an implanted reservoir. The term “parenteral” as used herein includes subcutaneous, intramuscular, intradermal, intravenous, intra-articular, intra-synovial, intrasternal, intrathecal, intrahepatic, intralesional and intracranial injection or infusion techniques. In a particular embodiment, the EP4 antagonist, antibody and/or anti-metabolite is administered orally. In another particular embodiment, the EP4 antagonist, antibody and/or antimetabolite is administered intravenously.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents, and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be un-coated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108 A, 4,166,452 A and 4,265,874 A to form osmotic therapeutic tablets for controlled release.

The following examples are illustrative of select embodiments of the present invention and are not meant to limit the scope of the invention.

### EXAMPLES

### Assays for Determining Biological Activity

The compounds of Formula I can be tested using the following assays to determine their prostanoid antagonist or agonist activity in vitro and in vivo and their selectivity. The prostaglandin receptor activities demonstrated are DP, EP₁, EP₂, EP₃, EP₄, FP, IP and TP.

### Example A. Stable Expression of Prostanoid Receptors in the Human Embryonic Kidney (HEK) 293(Ebna) Cell Line

Prostanoid receptor cDNAs corresponding to full length coding sequences are subcloned into appropriate sites of mammalian expression vectors and transfected into HEK 293(ebna) cells. HEK 293(ebna) cells expressing the individual cDNAs are grown under selection and individual colonies are isolated after 2-3 weeks of growth using a cloning ring-based method and subsequently expanded into clonal cell lines.

### Example B. Prostanoid Receptor Binding Assays

Transfected HEK 293(ebna) cells are maintained in culture and harvested, and membranes are prepared by differential centrifugation, following lysis of the cells in the presence of protease inhibitors, for use in receptor binding assays. Prostanoid receptor binding assays (for DP1, DP2 (CRTH2), EP1, EP2, EP3-III, EP4, FP, IP, and TP) are performed in 10 mM MES/KOH (pH 6.0) (EPs, FP and TP) or 10 mM HEPES/KOH (pH 7.4) (DPs and IP), containing 1 mM EDTA, 2.5-30 mM divalent cation and the appropriate radioligand that specifically binds to a target prostanoid receptor. Synthetic compounds are added in dimethyl sulfoxide which is kept constant at 1% (v/v) in all incubations. The reaction is initiated by addition of membrane protein. Non-specific binding is determined in the presence of 10 μM of the corresponding non-radioactive prostanoid. Incubations are conducted for 60-90 min at room temperature or 30° C. and terminated by rapid filtration. Compound binding is calculated as a % inhibition of the binding of the radioligand after subtracting nonspecific binding from total binding. The residual specific binding at each ligand concentration is calculated and expressed as a function of ligand concentration to construct sigmoidal concentration-response curve. Half maximal inhibitory concentration, IC₅₀ values of compounds are determined by nonlinear regression analysis of the concentration-response curves using Hill equation curve fitting equation Y=D+[(A−D)/(1+C/IC50)^{H}], where Y=specific binding, A=left asymptote of the curve, D=right asymptote of the curve, C=compound concentration, and H=slope factor. The binding affinity of the compounds is determined by calculating the equilibrium inhibition constant (Kᵢ) from the equation Kᵢ═InPt/1+[radioligand]/K_{d} where K_{d} is the equilibrium dissociation constant for the radioligand: receptor interaction and InPt is the inflection point of the dose-response curves.

EP4 receptor binding assays were performed at MSD Pharma Service in Taiwan under the following assay conditions:
Source: Human recombinant Chem-1 cells
Ligand: 1 nM [³H] Prostaglandin E₂ (PGE₂)
Vehicle: 1% DMSO
Incubation Time/Temp: 2 hours @25° C.
Incubation Buffer: 10 mM MES, pH 6.0, 1 mM EDTA, 10 mM MgCl₂
Non-Specific Ligand: 10 μM Prostaglandin E₂ (PGE₂)
K_{D}: 0.69 nM
Bₘₐₓ: 4.3 pmole/mg Protein
Specific binding: 90%
Quantitation Method: radioligand binding
Significance Criteria: >50% maximum inhibition

TABLE 1
Inhibition of PGE2 Binding of Representive Compounds

| Compound | | | IC₅₀ |
|---|---|---|---|
| ID | Compound Structure | Compound Name | (nM) |
| Example 1 | 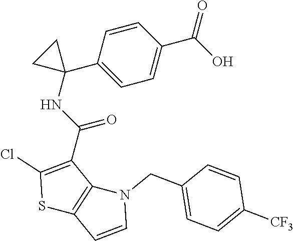 [SMILES: O=C(O)c1ccc(C2(NC(=O)c3c(Cl)sc4ccn(Cc5ccc(C(F)(F)F)cc5)c34)CC2)cc1] | 4-[1-[[2-chloro-4-[[4- (trifluoromethyl)phenyl]methyl]thieno [3,2-b]pyrrole-3- carbonyl]amino]cyclopropyl]benzoic acid | 0.25 |
| Example 2 | 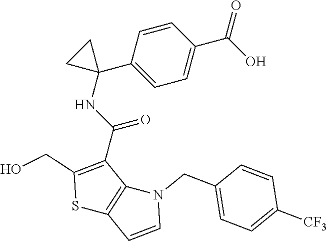 [SMILES: O=C(O)c1ccc(C2(NC(=O)c3c(CO)sc4ccn(Cc5ccc(C(F)(F)F)cc5)c34)CC2)cc1] | 4-[1-[[2-(hydroxymethyl)-4-[[4- (trifluoromethyl)phenyl]methyl]thieno [3,2-b]pyrrole-3- carbonyl]amino]cyclopropyl]benzoic acid | 1.1 |
| Example 3 | 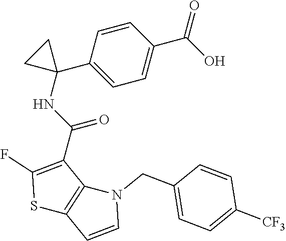 [SMILES: O=C(O)c1ccc(C2(NC(=O)c3c(F)sc4ccn(Cc5ccc(C(F)(F)F)cc5)c34)CC2)cc1] | 4-(1-(2-fluoro-4-(4- (trifluoromethyl)benzyl)-4H-thieno[3,2- b]pyrrole-3- carboxamido)cyclopropyl)benzoic acid | 0.48 |
| Example 4 | 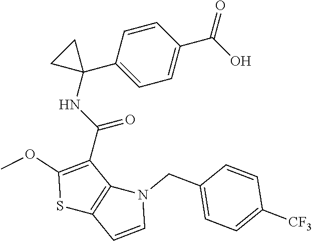 [SMILES: COc1sc2ccn(Cc3ccc(C(F)(F)F)cc3)c2c1C(=O)NC1(c2ccc(C(=O)O)cc2)CC1] | 4-(1-(2-methoxy-4-(4- (trifluoromethyl)benzyl)-4H-thieno[3,2- b]pyrrole-3- carboxamido)cyclopropyl)benzoic acid | 0.35 |
| Example 5 | 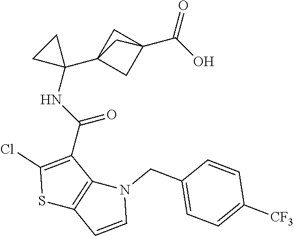 [SMILES: O=C(NC1(C23CC(C(=O)O)(C2)C3)CC1)c1c(Cl)sc2ccn(Cc3ccc(C(F)(F)F)cc3)c12] | 3-[1-[[2-chloro-4-[[4- (trifluoromethyl)phenyl]methyl]thieno[3, 2-b]pyrrole-3- carbonyl]amino]cyclopropyl]bicyclo [1.1.1]pentane-1-carboxylic acid | 28 |
| Example 6 | 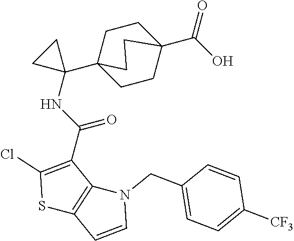 [SMILES: O=C(NC1(C23CCC(C(=O)O)(CC2)CC3)CC1)c1c(Cl)sc2ccn(Cc3ccc(C(F)(F)F)cc3)c12] | 4-[1-[[2-chloro-4-[[4- (trifluoromethyl)phenyl]methyl]thieno[3, 2-b]pyrrole-3- carbonyl]amino]cyclopropyl]bicyclo[2.2. 2]octane-1-carboxylic acid | 8.4 |
| Example 7 | 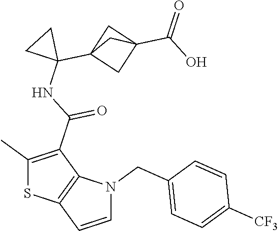 [SMILES: Cc1sc2ccn(Cc3ccc(C(F)(F)F)cc3)c2c1C(=O)NC1(C23CC(C(=O)O)(C2)C3)CC1] | 3-[1-[[2-methyl-4-[[4- (trifluoromethyl)phenyl]methyl]thieno[3, 2-b]pyrrole-3- carbonyl]amino]cyclopropyl]bicyclo[1.1. 1]pentane-1-carboxylic acid | 50 |
| Example 8 | 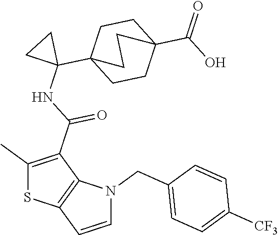 [SMILES: Cc1sc2ccn(Cc3ccc(C(F)(F)F)cc3)c2c1C(=O)NC1(C23CCC(C(=O)O)(CC2)CC3)CC1] | 4-[1-[[2-chloro-4-[[4- (trifluoromethyl)phenyl]methyl]thieno[3, 2-b]pyrrole-3- carbonyl]amino]cyclopropyl]bicyclo[2.2. 2]octane-1-carboxylic acid | 4.9 |
| Example 9 | 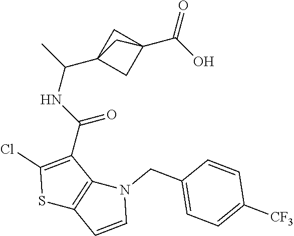 [SMILES: CC(NC(=O)c1c(Cl)sc2ccn(Cc3ccc(C(F)(F)F)cc3)c12)C12CC(C(=O)O)(C1)C2] | 3-[1-[[2-chloro-4-[[4- (trifluoromethyl)phenyl]methyl]thieno [3,2-b]pyrrole-3- carbonyl]amino]ethyl]bicyclo[1.1.1] pentane-1-carboxylic acid | 180 |
| Example 10 | 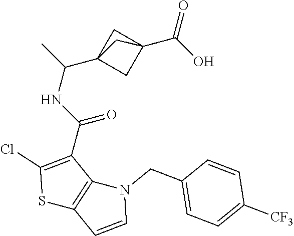 [SMILES: CC(NC(=O)c1c(Cl)sc2ccn(Cc3ccc(C(F)(F)F)cc3)c12)C12CC(C(=O)O)(C1)C2] | 3-[1-[[2-methyl-4-[[4- (trifluoromethyl)phenyl]methyl]thieno [3,2-b]pyrrole-3- carbonyl]amino]ethyl]bicyclo[1.1.1] pentane-1-carboxylic acid | 620 |
| Example 11 | 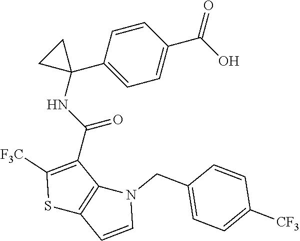 [SMILES: O=C(O)c1ccc(C2(NC(=O)c3c(C(F)(F)F)sc4ccn(Cc5ccc(C(F)(F)F)cc5)c34)CC2)cc1] | 4-(1-(2-(trifluoromethyl)-4-(4- (trifluoromethyl)benzyl)-4H-thieno[3,2- b]pyrrole-3- carboxamido)cyclopropyl)benzoic acid | 1.2 |

### Example C. Microsomal Stability Assay

Microsomal stability assay is performed to understand the metabolism of the representative compounds.

In vitro microsomal stability was determined in pooled human, dog, rat, and mouse liver microsomes. Test compounds (Table 2) were incubated in the presence and absence of NADPH for 0-60 min and the amount of remaining compound was quantified through LC-MS/MS analysis.

TABLE 2
Liver microsomal stability of Representative Compounds

| | | | In vitro CLᵢₙₜ | |
|---|---|---|---|---|
| Compound | | In vitro T_{1/2} | (μL/min/mg | Scale-up CLᵢₙₜ |
| ID | Species | (min) | protein) | (mL/min/kg) |
| Example 1 | Human | 122 | 11.4 | 14.3 |
| | Dog | 140 | 9.89 | 24.6 |
| | Rat | 104 | 13.4 | 24.0 |
| | Mouse | 92.3 | 15.0 | 66.1 |
| Example 3 | Human | 344 | 4.03 | 5.05 |
| | Dog | 434 | 3.20 | 7.97 |
| | Rat | 165 | 8.40 | 15.0 |
| | Mouse | 202 | 6.85 | 30.1 |
| Example 4 | Human | 428 | 3.24 | 4.06 |
| | Dog | 229 | 6.06 | 15.1 |
| | Rat | 125 | 11.1 | 19.8 |
| | Mouse | 131 | 10.6 | 46.7 |

### Example D. Pharmacokinetic (PK) Assay

Test compound was dissolved in 10% DMSO, 40% PEG-400, and 50% water for intravenous (i.v.) and oral (p.o.) administration to rat. Three male Sprague-Dawley rats received single imv. doses into the caudal vein (2 mg/kg). Three male animals received single p.o. doses by gavage tube (5 mg/kg). Blood samples were collected into K2-EDTA tubes from the jugular vein at 0.083, 0.25, 0.5, 1, 2, 4, 8, and 24 h post dosing. After centrifugation, plasma samples were prepared via protein precipitation and detected through LC-MS/MS. The pharmacokinetics parameters were calculated using Phoenix software V 8.3.4.

TABLE 3
Rat PK Assay of Example 1

Example 1

| | | |
|---|---|---|
| Rat iv | Dose (mg/kg) | 5 |
| | T_{1/2} (hr) | 9.47 |
| | C0 (ng/mL) | 8385 |
| | AUC₀₋ₜ (ng · hr/mL) | 27712 |
| | AUC_{0-∞} (ng · hr/mL) | 33593 |
| | Vss_obs (L/kg) | 1.64 |
| | CL_obs(mL/min/kg) | 2.57 |
| | MRT (hr) | 5.93 |
| Rat po | Dose (mg/kg) | 10 |
| | T_{1/2} (hr) | 8.97 |
| | Tmax (hr) | 6.67 |
| | Cmax (ng/mL) | 1830 |
| | AUC₀₋ₜ (ng · hr/mL) | 23419 |
| | AUC_{0-∞} (ng · hr/mL) | 26729 |
| | MRT (hr) | 7.98 |
| | F % | 42.3 |

### Example E. Prostanoid Receptor Agonist and Antagonist Assays

Whole cell second messenger assays measuring stimulation of intracellular cAMP accumulation in HEK-293(ebna)-hEP4 cells are performed to determine whether receptor ligands are agonists or antagonists. Cells are harvested and resuspended in HBSS containing 25 mM HEPES, pH 7.4. Incubations contain 0.5 mM IBMX (phosphodiesterase inhibitor, available from Biomol). Samples are incubated at 37° C. for 10 min, the reaction is terminated, and cAMP levels are then measured. Ligands are added in dimethylsulfoxide which is kept constant at 1% (v/v; agonists) or 2% (v/v; antagonists) in all incubations. For agonists, second messenger responses are expressed as a function of ligand concentration and both EC₅₀ values and the maximum response as compared to a PGE2 standard are calculated. For antagonists, the ability of a ligand to inhibit an agonist response is determined by carrying out dose-response curves in the presence of PGE22 agonist at a concentration corresponding to its EC₇₀. IC₅₀ values are calculated as the concentration of ligand required to inhibit 50% of the PGE2-induced activity.

### Example F. Rat Paw Edema Assay

The method is the same as described in Chan et al (J. Pharmacol. Exp. Ther. 274: 1531-1537, 1995).

### Example G. Acute Inflammatory Hyperalgesia Induced by Carrageenan in Rats

The method is the same as described in Boyce et al (Neuropharmacology 33: 1609-1611, 1994).

### Example H. Adjuvant-Induced Arthritis in Rats

Female Lewis rats (body weight ˜146-170 g) are weighed, ear marked, and assigned to groups (a negative control group in which arthritis was not induced, a vehicle control group, a positive control group administered indomethacin at a total daily dose of 1 mg/kg and four groups administered with a test compound at total daily doses of 0.10-3.0 mg/kg) such that the body weights were equivalent within each group. Six groups of 10 rats each are injected into a hind paw with 0.5 mg of Mycobacterium butyricum in 0.1 mL of light mineral oil (adjuvant), and a negative control group of 10 rats was not injected with adjuvant. Body weights, contralateral paw volumes (determined by mercury displacement plethysmography) and lateral radiographs (obtained under Ketamine and Xylazine anesthesia) are determined before (day −1) and 21 days following adjuvant injection, and primary paw volumes are determined before (day −1) and on days 4 and 21 following adjuvant injection. The rats are anesthetized with an intramuscular injection of 0.03-0.1 mL of a combination of Ketamine (87 mg/kg) and Xylazine (13 mg/kg) for radiographs and injection of adjuvant. The radiographs are made of both hind paws on day 0 and day 21 using the Faxitron (45 kVp, 30 seconds) and Kodak X-OMAT TL film, and are developed in an automatic processor. Radiographs are evaluated for changes in the soft and hard tissues by an investigator who was blinded to experimental treatment. The following radiographic changes are graded numerically according to severity: increased soft issue volume (0-4), narrowing or widening of joint spaces (0-5) subchondral erosion (0-3), periosteal reaction (0-4), osteolysis (0-4) subluxation (0-3), and degenerative joint changes (0-3). Specific criteria are used to establish the numerical grade of severity for each radiographic change. The maximum possible score per foot was 26. A test compound at total daily doses of 0.1, 0.3, 1, and 3 mg/kg/day, indomethacin at a total daily dose of 1 mg/kg/day, or vehicle (0.5% Methocel™ in sterile water) are administered per os b.i.d. beginning post injection of adjuvant and continuing for 21 days. The compounds are prepared weekly, refrigerated in the dark until used, and vortex mixed immediately prior to administration.

### Example I. Mouse Syngeneic Model for Measurement of Antitumor Activity

The assays described in the article by Spranger et al (Journal for ImmunoTherapy of Cancer, 2014, 2:3) can be used to evaluate the synergistic effects of the compounds of the present invention in combination with an effective amount of antibodies to cytotoxic t-lymphocyte antigen 4 (anti-CTLA4); antibodies to programmed death ligand 1 (anti-PD L1); antibodies to programmed cell death protein 1 (anti-PD1); indoleamine-2,3-dioxygenase (IDO) inhibitors; tryptophan-2,3-dioxygenase (TDO) inhibitors.

### Synthesis of Compounds

### Example 1: 4-[1-[[2-chloro-4-[[4-(trifluoromethyl)phenyl]methyl]thieno[3,2-b]pyrrole-3-carbonyl]amino]cyclopropyl]benzoic acid

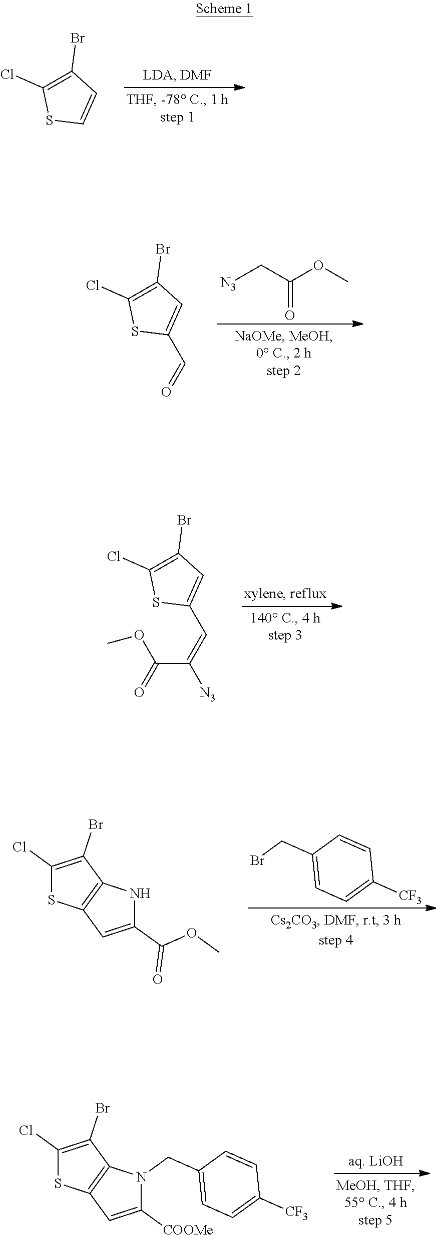

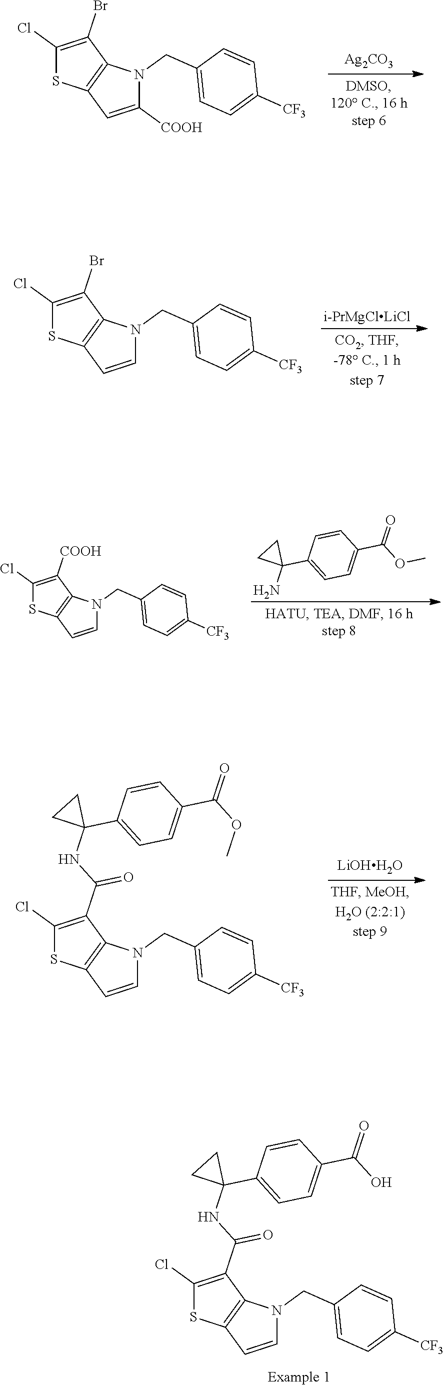

### Step 1: 4-bromo-5-chloro-thiophene-2-carbaldehyde

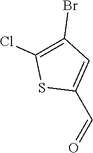

To a solution of 3-bromo-2-chloro-thiophene (5 g, 25.32 mmol, 1 eq) in THF (50 mL) was added LDA (2.5 M, 15.19 mL, 1.5 eq) dropwise over 45 minutes under argon at −78° C. The reaction mixture was stirred for one hour at −78° C., then dry DMF (9.25 g, 126.59 mmol, 9.74 mL, 5 eq) was added over 15 minutes at −78° C., after the addition was finished, warmed to 25° C., and stirred for 45 minutes. The reaction was monitored by TLC, after the reaction was finished, aqueous citric acid was added and the reaction mixture was stirred for 5 minutes. The mixture was diluted with ethyl acetate and washed with brine. The organic layer was dried over magnesium sulfate, filtered, concentrated in vacuo. The residue was purified by column chromatography (SiO₂, Petroleum ether/Ethyl acetate=5/1). Compound 4-bromo-5-chloro-thiophene-2-carbaldehyde (4.7 g, 20.84 mmol, 82.3% yield) was obtained as a yellow solid.

### Step 2: methyl (E)-2-azido-3-(4-bromo-5-chloro-2-thienyl)prop-2-enoate

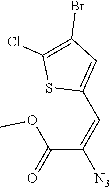 [SMILES: COC(=O)/C(=C\c1cc(Br)c(Cl)s1)N=[N+]=[N-]]

To a solution of 4-bromo-5-chloro-thiophene-2-carbaldehyde (18 g, 79.83 mmol, 1 eq), methyl 2-azidoacetate (27.56 g, 239.48 mmol, 3 eq) in MeOH (180 mL) was added a solution of NaOMe in MeOH (4.36 M, 18.31 mL, 1 eq) at 0° C., after the addition was finished, the mixture was stirred at 0° C. for 2 hr. The reaction was monitored by TLC, after the reaction was finished, the reaction was quenched by the addition of saturated NH₄Cl at 25° C., and then diluted with Water, extracted with ethyl acetate (50 mL×3), the organic layer was collected and dried over Na₂SO₄, filtered and concentrated under reduced pressure to give the crude methyl (E)-2-azido-3-(4-bromo-5-chloro-2-thienyl)prop-2-enoate (17 g, 52.70 mmol, 66.0% yield) as a yellow solid which was used directly in the next step without further purification.

### Step 3: methyl 3-bromo-2-chloro-4H-thieno[3,2-b]pyrrole-5-carboxylate

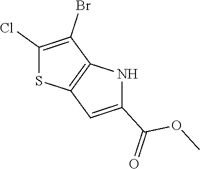

A mixture of methyl (E)-2-azido-3-(4-bromo-5-chloro-2-thienyl)prop-2-enoate (2.3 g, 7.13 mmol, 1 eq) in xylene (30 mL) was degassed and purged with N₂ for 3 times, and then the mixture was stirred at 140° C. for 4 hr under N₂ atmosphere. The reaction was monitored by TLC, after the reaction was finished, the solution was cooled to room temperature and stand overnight, the solid was collected by filtration and dried in vacuo to give methyl 3-bromo-2-chloro-4H-thieno[3,2-b]pyrrole-5-carboxylate (1.5 g, 5.09 mmol, 71.4% yield) as a white solid which was used in the next step without further purification.

### Step 4: methyl 3-bromo-2-chloro-4-[[4-(trifluoromethyl)phenyl]methyl]thieno[3,2-b]pyrrole-5-carboxylate

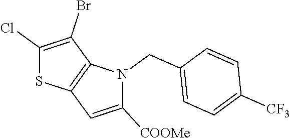 [SMILES: COC(=O)c1cc2sc(Cl)c(Br)c2n1Cc1ccc(C(F)(F)F)cc1]

To a solution of methyl 3-bromo-2-chloro-4H-thieno[3,2-b]pyrrole-5-carboxylate (1.42 g, 4.82 mmol, 1 eq), 1-(bromomethyl)-4-(trifluoromethyl)benzene (1.15 g, 4.82 mmol, 743.45 uL, 1 eq) in DMF (15 mL) was added Cs₂CO₃ (4.71 g, 14.46 mmol, 3 eq). The mixture was stirred at 25° C. for 3 hr. The reaction was monitored by TLC, after the reaction was finished, the reaction mixture was diluted with water and extracted with EtOAc (30 mL×3). The combined organic layers were washed with brine (2 mL×3), dried over Na₂SO₄, filtered and concentrated under reduced pressure. The residue was purified by column chromatography (SiO₂, Petroleum ether/Ethyl acetate=10/1) to give methyl 3-bromo-2-chloro-4-[[4-(trifluoromethyl)phenyl]methyl]thieno[3,2-b]pyrrole-5-carboxylate (1.93 g, 4.26 mmol, 88.4% yield) as a white solid. MS (ES-API positive): 453.9 (M+2)⁺.

### Step 5: 3-bromo-2-chloro-4-[[4-(trifluoromethyl)phenyl]methyl]thieno[3,2-b]pyrrole-5-carboxylic acid

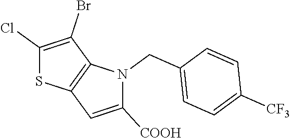 [SMILES: O=C(O)c1cc2sc(Cl)c(Br)c2n1Cc1ccc(C(F)(F)F)cc1]

To a solution of the methyl 3-bromo-2-chloro-4-[[4-(trifluoromethyl)phenyl]methyl]thieno[3,2-b]pyrrole-5-carboxylate (1 g, 2.21 mmol, 1 eq) in THF (12.5 mL) and MeOH (12.5 mL) was added LiOH·H₂O (1 M, 6.63 mL, 3 eq) (1N aqueous solution). The reaction mixture was stirred at 55° C. for 4 h. The reaction was monitored by LCMS, after the reaction was finished, most of the solvent was removed by concentration, the residue was diluted with water and acidified with 1 N HCl to pH=3-4, extracted with EtOAc (15 mL×3). The combined organic layers were dried over Na₂SO₄, filtered and concentrated in vacuum to give 3-bromo-2-chloro-4-[[4-(trifluoromethyl)phenyl]methyl]thieno[3,2-b]pyrrole-5-carboxylic acid (950 mg, 2.17 mmol, 98.04% yield) as a white solid which was used in the next step without further purification. MS (ES-API positive): 439.0 (M+2)⁺.

### Step 6: 3-bromo-2-chloro-4-[[4-(trifluoromethyl)phenyl]methyl]thieno[3,2-b]pyrrole

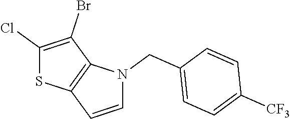 [SMILES: FC(F)(F)c1ccc(Cn2ccc3sc(Cl)c(Br)c32)cc1]

To a solution of 3-bromo-2-chloro-4-[[4-(trifluoromethyl)phenyl]methyl]thieno[3,2-b]pyrrole-5-carboxylic acid (800 mg, 1.82 mmol, 1 eq) in DMSO (8 mL) was added Ag₂CO₃ (502.90 mg, 1.82 mmol, 82.71 uL, 1 eq) and 2-3 drops of HOAc. The mixture was stirred at 120° C. under N₂ for 16 hr. The reaction was monitored by LCMS, after the reaction was finished, the reaction mixture was diluted with H₂O (15 mL) and extracted with Ethyl acetate (20 mL×3). The combined organic layers were washed with brine, dried over Na₂SO₄, filtered and the filtrate was concentrated under reduced pressure to give 3-bromo-2-chloro-4-[[4-(trifluoromethyl)phenyl]methyl]thieno[3,2-b]pyrrole (650 mg, 1.65 mmol, 90.31% yield) as a light yellow solid which was used into the next step without further purification.

MS (ES-API positive): 396.0 (M+2)⁺.

### Step 7: 2-chloro-4-[[4-(trifluoromethyl)phenyl]methyl]thieno[3,2-b]pyrrole-3-carboxylic acid

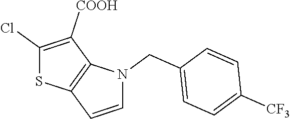 [SMILES: O=C(O)c1c(Cl)sc2ccn(Cc3ccc(C(F)(F)F)cc3)c12]

To a mixture of 3-bromo-2-chloro-4-[[4-(trifluoromethyl)phenyl]methyl]thieno[3,2-b]pyrrole (500 mg, 1.27 mmol, 1 eq) in THF (1.5 mL) was added lithium; chloro(isopropyl)magnesium; chloride (1.3 M, 1.27 mL, 1.3 eq) at −78° C. under N₂. The reaction mixture was stirred at −78° C. for 30 min. Then dry CO₂ was bubbled into the reaction mixture for 20 min at −78° C. The reaction was monitored by LCMS, after the reaction was finished, the reaction was quenched by the addition of water (8 mL), then extracted with EtOAc (10 mL×3). The organic layer was dried over Na₂SO₄, filtered and concentrated in vacuum. Crude 2-chloro-4-[[4-(trifluoromethyl)phenyl]methyl]thieno[3,2-b]pyrrole-3-carboxylic acid (460 mg, 767.20 μmol, 60.55% yield, 60% purity) was obtained as a light-yellow oil, which was used directly to next step without further purification.

MS (ES-API positive): 360.0 (M+1)⁺.

### Step 8: methyl 4-[1-[[2-chloro-4-[[4-(trifluoromethyl)phenyl]methyl]thieno[3,2-b]pyrrole-3-carbonyl]amino]cyclopropyl]benzoate

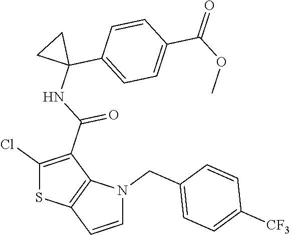 [SMILES: COC(=O)c1ccc(C2(NC(=O)c3c(Cl)sc4ccn(Cc5ccc(C(F)(F)F)cc5)c34)CC2)cc1]

To a mixture of 2-chloro-4-[[4-(trifluoromethyl)phenyl]methyl]thieno[3,2-b]pyrrole-3-carboxylic acid (5.35 g, 14.87 mmol, 1 eq) and TEA (4.51 g, 44.61 mmol, 6.21 mL, 3 eq) in DMF (60 mL) was added HATU (6.79 g, 17.85 mmol, 1.2 eq). The reaction mixture was stirred at room temperature for 30 min. Then methyl 4-(1-aminocyclopropyl)benzoate (2.84 g, 14.87 mmol, 1 eq) was added to the reaction mixture, then stirred at room temperature overnight. The reaction was monitored by LCMS, after the reaction was finished, most of the solvent DMF was removed by concentration, the residue was diluted with EtOAc (50 mL), the solid was collected by filtration and washed with EtOAc (10 mL), then dried over vacuum to give methyl 4-[1-[[2-chloro-4-[[4-(trifluoromethyl)phenyl]methyl]thieno[3,2-b]pyrrole-3-carbonyl]amino]cyclopropyl]benzoate (5.1 g, 9.57 mmol, 64.35% yield) as a white solid which was used directly to next step.

MS (ES-API positive): 533.1 (M+1)⁺.

¹H-NMR (500 MHz, DMSO-d6) δ 9.33 (s, 1H), 7.73 (d, J=6.4 Hz, 2H), 7.64 (br d, J=6.4 Hz, 2H), 7.27-7.20 (m, 3H), 7.07 (br d, J=6.4 Hz, 2H), 6.50 (d, J=2.4 Hz, 1H), 5.45 (s, 2H), 3.82 (s, 3H), 1.26-1.20 (m, 2H), 0.99-0.93 (m, 2H).

### Step 9: 4-[1-[[2-chloro-4-[[4-(trifluoromethyl)phenyl]methyl]thieno[3,2-b]pyrrole-3-carbonyl]amino]cyclopropyl]benzoic acid

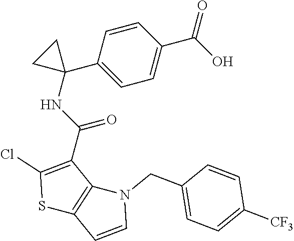 [SMILES: O=C(O)c1ccc(C2(NC(=O)c3c(Cl)sc4ccn(Cc5ccc(C(F)(F)F)cc5)c34)CC2)cc1]

A mixture of methyl 4-[1-[[2-chloro-4-[[4-(trifluoromethyl)phenyl]methyl]thieno[3,2-b]pyrrole-3-carbonyl]amino]cyclopropyl]benzoate (5.1 g, 9.57 mmol, 1 eq) in THF (60 mL) and H₂O (40 mL) was added LiOH·H₂O (4.02 g, 95.69 mmol, 10 eq). The reaction mixture was stirred at 50° C. for 16 hours. The reaction was monitored by LCMS, after the reaction was finished, most of the solvent was removed by concentration, then diluted with water and acidified with 1N HCl to pH˜4. Then it was extracted with EtOAc (200 mL×3), the combined organic layers were dried over Na₂SO₄, filtered and concentrated. The residue was recrystallized in EtOAc (40 mL) to give 4-[1-[[2-chloro-4-[[4-(trifluoromethyl)phenyl]methyl]thieno[3,2-b]pyrrole-3-carbonyl]amino]cyclopropyl]benzoic acid (3.8 g, 7.32 mmol, 76.52% yield, 100% purity) as a light yellow solid.

¹H-NMR (400 MHz, DMSO-d6) δ 12.80 (br s, 1H), 9.33 (s, 1H), 7.74 (d, J=6.4 Hz, 2H), 7.63 (d, J=6.4 Hz, 2H), 7.30-7.18 (m, 3H), 7.07 (d, J=6.4 Hz, 2H), 6.50 (d, J=2.4 Hz, 1H), 5.45 (s, 2H) 1.25-1.18 (m, 2H), 0.96-0.90 (m, 2H).

MS (ES-API positive): 519.2 (M+1)⁺.

### Step 10: sodium 4-(1-(2-chloro-4-(4-(trifluoromethyl)benzyl)-4H-thieno[3,2-b]pyrrole-3-carboxamido)cyclopropyl)benzoate

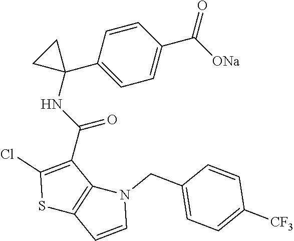

To a solution of methyl 4-[1-[[2-chloro-4-[[4-(trifluoromethyl)phenyl]methyl]thieno[3,2-b]pyrrole-3-carbonyl]amino]cyclopropyl]benzoate (26 g, 48.78 mmol, 1 eq) in THF (200 mL), H₂O (100 mL) and MeOH (50 mL) was added NaOH (5.85 g, 146.35 mmol, 3 eq) at 20° C., then stirred at 50° C. for 16 hr. The reaction was monitored by TLC and LCMS, after the reaction was finished, most of THF was removed by concentration, water (100 mL) was added, after stirred at room temperature for 2 h, the solid was collected by filtration, the filter cake was washed with water (100 mL×2), then dried in vacuo to give sodium 4-(1-(2-chloro-4-(4-(trifluoromethyl)benzyl)-4H-thieno[3,2-b]pyrrole-3-carboxamido)cyclopropyl)benzoate (25 g, 46.22 mmol, 94.74% yield) as pale yellow solid. ¹H-NMR (400 MHz, DMSO-d6) δ 7.68 (d, J=6.8 Hz, 2H), 7.62 (d, J=6.4 Hz, 2H), 7.27 (d, J=6.8 Hz, 2H), 7.08 (d, J=2.4 Hz, 1H), 6.98 (d, J=2.4 Hz, 2H), 6.43 (d, J=2.4 Hz, 1H), 5.41 (s, 2H), 1.18 (t, J=7.2 Hz, 2H), 0.93 (t, J=7.2 Hz, 2H).

MS (ES-API positive): 519.0 (M+1)⁺.

### Example 2

### 4-[1-[[2-(hydroxymethyl)-4-[[4-(trifluoromethyl)phenyl]methyl]thieno[3,2-b]pyrrole-3-carbonyl]amino]cyclopropyl]benzoic acid

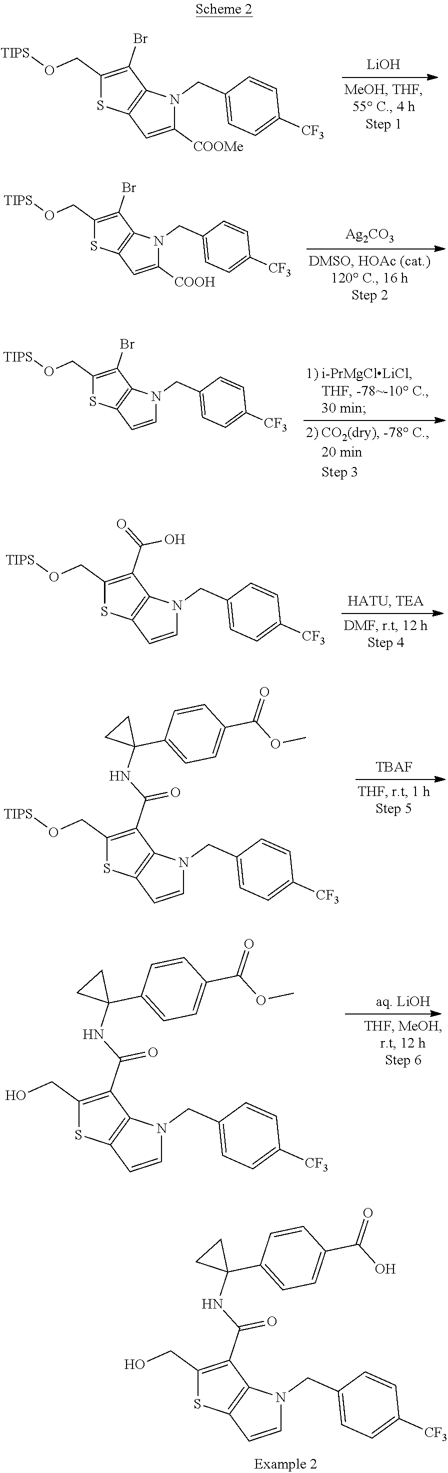

### Step 1: 3-bromo-2-chloro-4-[[4-(trifluoromethyl)phenyl]methyl]thieno[3,2-b]pyrrole-5-carboxylic acid

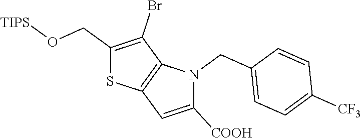

To a solution of the methyl 3-bromo-2-chloro-4-[[4-(trifluoromethyl)phenyl]methyl]thieno[3,2-b]pyrrole-5-carboxylate (1 g, 2.21 mmol, 1 eq) in THF (12.5 mL) and MeOH (12.5 mL) was added 1 M LiOH (6.63 mL, 3 eq). The reaction mixture was stirred at 55° C. for 4 h. The reaction was monitored by LCMS, after the reaction was finished, most of the solvent was removed by concentration, then diluted with water, and acidified with 1 N HCl to pH=3-4, extracted with EtOAc (15 mL×3). The combined organic layers were dried over Na₂SO₄, filtered and concentrated in vacuum to give the crude 3-bromo-2-chloro-4-[[4-(trifluoromethyl)phenyl]methyl]thieno[3,2-b]pyrrole-5-carboxylic acid (950 mg, 2.17 mmol, 98.04% yield) as a white solid. MS (ES-API positive): 620.4 (M+1)⁺.

### Step 2: 3-bromo-2-chloro-4-[[4-(trifluoromethyl)phenyl]methyl]thieno[3,2-b]pyrrole

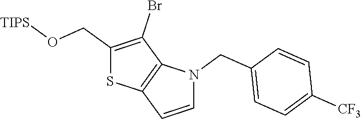

To a solution of 3-bromo-2-chloro-4-[[4-(trifluoromethyl)phenyl]methyl]thieno[3,2-b]pyrrole-5-carboxylic acid (800 mg, 1.82 mmol, 1 eq) in DMSO (8 mL) was added Ag₂CO₃ (502.90 mg, 1.82 mmol, 82.71 μL, 1 eq) and 50 mg of HOAc. The mixture was stirred at 120° C. under N2 for 16 hr. The reaction was monitored by LCMS, after the reaction was finished, diluted with H₂O (50 mL) and extracted with Ethyl acetate (20 mL×3). The combined organic layers were washed with brine, dried over Na₂SO₄, filtered and the filtrate was concentrated under reduced pressure to give 3-bromo-2-chloro-4-[[4-(trifluoromethyl)phenyl]methyl]thieno[3,2-b]pyrrole (650 mg, 1.65 mmol, 90.31% yield) as a light yellow solid, which was used into the next step without further purification. MS (ES-API positive): 396.0 (M+1)+

### Step 3: 4-[[4-(trifluoromethyl)phenyl]methyl]-2-(triisopropylsilyloxymethyl)thieno[3,2-b]pyrrole-3-carboxylic acid

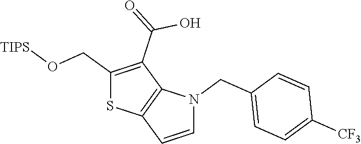

To a solution of [3-bromo-4-[[4-(trifluoromethyl)phenyl]methyl]thieno[3,2-b]pyrrol-2-yl]methoxy-triisopropyl-silane (350 mg, 640.37 μmol, 1 eq) in THF (7 mL) was added lithium; chloro(isopropyl)magnesium (1.3 M, 985.19 μL, 2 eq) at −78° C. over 5 minutes under N₂, after the addition was finished, the reaction mixture was warmed to −10° C. and stirred at −10° C. for 30 min, then cooled back to −78° C., then dry CO₂ was bubbled into the reaction mixture for 20 min at −78° C. The reaction was monitored by LCMS, after the reaction was finished, the reaction was quenched by the addition of water, extracted with EtOAc (10 mL×3). The organic layer was dried over Na₂SO₄, filtered and concentrated in vacuum to give 4-[[4-(trifluoromethyl)phenyl]methyl]-2-(triisopropylsilyloxymethyl)thieno[3,2-b]pyrrole-3-carboxylic acid (326 mg, 637.13 μmol, 99.49% yield) as a yellow solid.

MS (ES-API positive): 338.0 (M+1)⁺.

### Step 4: methyl 4-[1-[[4-[[4-(trifluoromethyl)phenyl]methyl]-2-(triisopropylsilyloxymethyl)thieno[3,2-b]pyrrole-3-carbonyl]amino]cyclopropyl]benzoate

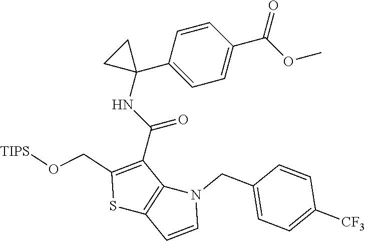

To a solution of 4-[[4-(trifluoromethyl)phenyl]methyl]-2-(triisopropylsilyloxymethyl)thieno[3,2-b]pyrrole-3-carboxylic acid (210 mg, 410.42 μmol, 1 eq) in DMF (4 mL) was added HATU (202.87 mg, 533.55 μmol, 1.3 eq) and TEA (83.06 mg, 820.84 μmol, 114.25 μL, 2 eq). The reaction mixture was stirred at 25° C. for 30 min. Then methyl 4-(1-aminocyclopropyl)benzoate (78.48 mg, 410.42 μmol, 1 eq) was added to the reaction mixture and stirred at 25° C. for 12 h. The reaction was monitored by LCMS, after the reaction was finished, the reaction mixture was diluted with EtOAc (60 mL), washed with brine (8 mL×5), dried over Na₂SO₄, then concentrated in vacuum to give methyl-4-[1-[[4-[[4-(trifluoromethyl)phenyl]methyl]-2-(triisopropylsilyloxymethyl)thieno[3,2-b]pyrrole-3-carbonyl]amino]cyclopropyl]benzoate (220 mg, 321.22 μmol, 78.27% yield) as a yellow oil, which was used directly to next step without further purification.

MS (ES-API positive): 707.3 (M+1)⁺.

### Step 5: methyl 4-[1-[[2-(hydroxymethyl)-4-[[4-(trifluoromethyl)phenyl]methyl]thieno[3,2-b]pyrrole-3-carbonyl]amino]cyclopropyl]benzoate

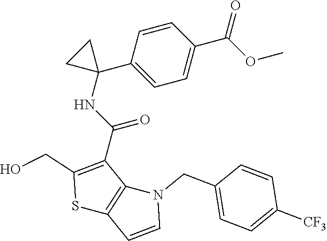 [SMILES: COC(=O)c1ccc(C2(NC(=O)c3c(CO)sc4ccn(Cc5ccc(C(F)(F)F)cc5)c34)CC2)cc1]

To a solution of methyl 4-[1-[[4-[[4-(trifluoromethyl)phenyl]methyl]-2-(triisopropylsilyloxymethyl)thieno[3,2-b]pyrrole-3-carbonyl]amino]cyclopropyl]benzoate (220 mg, 321.22 μmol, 1 eq) in THF (3 mL) was added TBAF (1 M, 385.47 μL, 1.2 eq). The reaction mixture was stirred at 25° C. for 1 h. TLC showed the starting material was consumed; the desired compound was observed. The reaction mixture was concentrated in vacuum. The residue was purified by column chromatography (SiO₂, petroleum ether:EtOAc=1:0 to 3:1) to give methyl 4-[1-[[2-(hydroxymethyl)-4-[[4-(trifluoromethyl)phenyl]methyl]thieno[3,2-b]pyrrole-3-carbonyl]amino]cyclopropyl]benzoate (140 mg, 264.88 μmol, 82.46% yield) as a light yellow solid.

MS (ES-API positive): 551.1 (M+1)⁺.

### Step 6: 4-[1-[[2-(hydroxymethyl)-4-[[4-(trifluoromethyl)phenyl]methyl]thieno[3,2-b]pyrrole-3-carbonyl]amino]cyclopropyl]benzoic acid

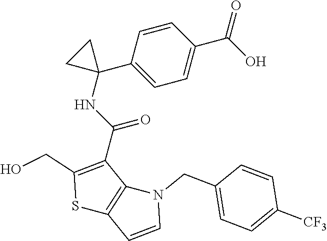 [SMILES: O=C(O)c1ccc(C2(NC(=O)c3c(CO)sc4ccn(Cc5ccc(C(F)(F)F)cc5)c34)CC2)cc1]

To a solution of methyl 4-[1-[[2-(hydroxymethyl)-4-[[4-(trifluoromethyl)phenyl]methyl]thieno[3,2-b]pyrrole-3-carbonyl]amino]cyclopropyl]benzoate (82 mg, 155.14 μmol, 1 eq) in THF (3 mL) and H2O (1 mL) was added LiOH·H₂O (32.55 mg, 775.72 μmol, 5 eq). The reaction mixture was stirred at room temperature for 12 h. The reaction was monitored by LCMS, after the reaction was finished, the reaction mixture was acidified with 1N HCl to pH˜4, extracted with EtOAc (12 mL×3). The organic layer was washed with brine (6 mL), dried over Na₂SO₄, filtered and concentrated. The residue was purified by prep-HPLC (TFA) (column: Boston Green ODS 150×30 mm×5 μm; mobile phase: [water(TFA)-ACN]; B %: 48%-68%, min) followed by lyophilization to give 4-[1-[[2-(hydroxymethyl)-4-[[4-(trifluoromethyl)phenyl]methyl]thieno[3,2-b]pyrrole-3-carbonyl]amino]cyclopropyl]benzoic acid (30.05 mg, 58.40 μmol, 37.65% yield) as a white solid.

¹H NMR (500 MHz, DMSO-d6) δ 12.98-12.59 (m, 1H), 9.06-8.93 (m, 1H), 7.75-7.66 (m, 2H), 7.64-7.56 (m, 2H), 7.21-7.10 (m, 3H), 7.07-6.98 (m, 2H), 6.52-6.42 (m, 1H), 5.57-5.50 (m, 2H), 4.77-4.68 (m, 2H), 1.28-1.15 (m, 2H), 1.09-0.99 (m, 2H).

MS (ES-API positive): 497.2 (M+1)⁺.

### Examples 3 and 4

### 4-(1-(2-fluoro-4-(4-(trifluoromethyl)benzyl)-4H-thieno[3,2-b]pyrrole-3-carboxamido) cyclopropyl)benzoic acid (Example 3) and 4-(1-(2-methoxy-4-(4-(trifluoromethyl)benzyl)-4H-thieno[3,2-b]pyrrole-3-carboxamido)cyclopropyl)benzoic acid (Example 4)

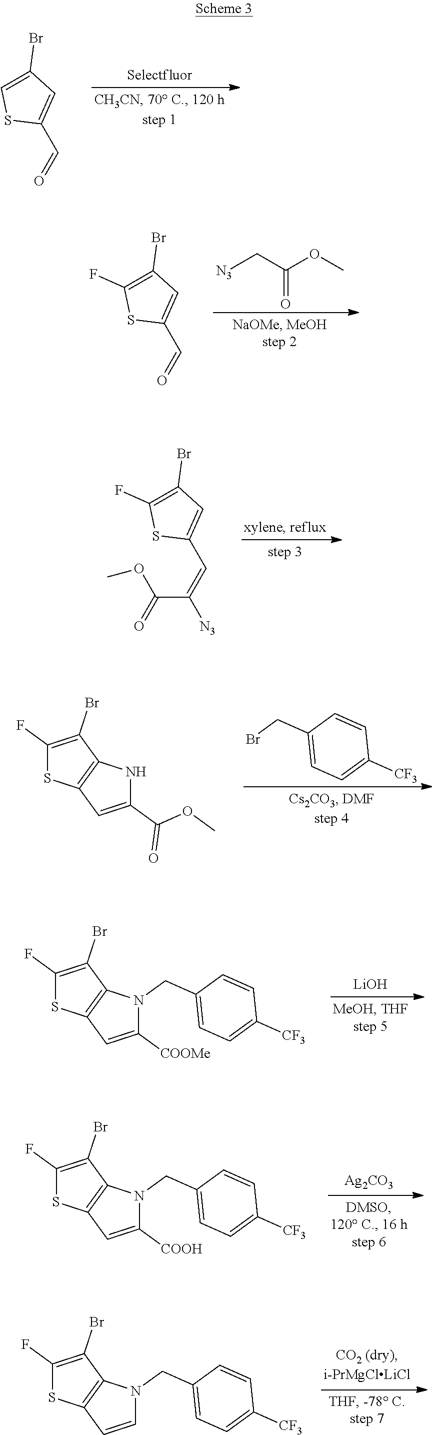

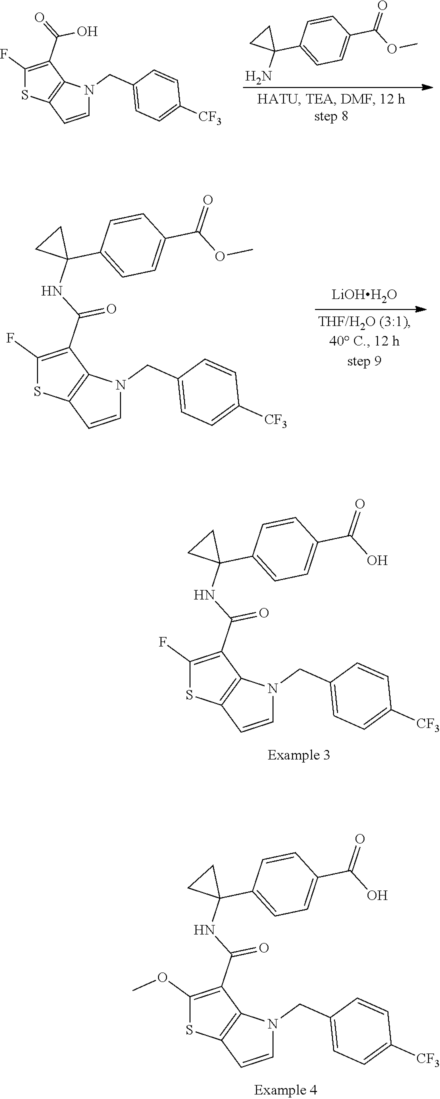 [SMILES: O=C(O)c1c(F)sc2ccn(Cc3ccc(C(F)(F)F)cc3)c12 ; COC(=O)c1ccc(C2(N)CC2)cc1 ; COC(=O)c1ccc(C2(NC(=O)c3c(F)sc4ccn(Cc5ccc(C(F)(F)F)cc5)c34)CC2)cc1 ; "LiOH·H2O" ; O=C(O)c1ccc(C2(NC(=O)c3c(F)sc4ccn(Cc5ccc(C(F)(F)F)cc5)c34)CC2)cc1 ; COc1sc2ccn(Cc3ccc(C(F)(F)F)cc3)c2c1C(=O)NC1(c2ccc(C(=O)O)cc2)CC1]

### Step 1: 4-bromo-5-fluorothiophene-2-carbaldehyde

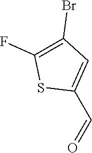

To a solution of 4-bromothiophene-2-carbaldehyde (7 g, 36.64 mmol, 1 eq) in CH₃CN (40 mL), water (40 mL) was added Select Fluor (19.47 g, 54.96 mmol, 1.5 eq). The mixture was stirred at 70° C. for 120 hr. The reaction was monitored by TLC, after the reaction was finished, most of the solvent was removed by concentration, the residue was diluted with Water and extracted with EtOAc (3×100 mL). The combined organic layers were washed with brine, dried over Na₂SO₄, filtered, and concentrated under reduced pressure. The residue was purified by flash silica gel chromatography (ISCO®; 80 g SepaFlash® Silica Flash Column, Eluent of 0˜20% Ethyl acetate/Petroleum ethergradient @20 mL/min) to give 4-bromo-5-fluoro-thiophene-2-carbaldehyde (5 g, 23.92 mmol, 65.28% yield) as a white solid.

### Step 2: methyl (E)-2-azido-3-(4-bromo-5-fluorothiophen-2-yl) acrylate

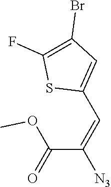 [SMILES: COC(=O)/C(=C\c1cc(Br)c(F)s1)N=[N+]=[N-]]

To a solution of 4-bromo-5-fluoro-thiophene-2-carbaldehyde (4 g, 19.14 mmol, 1 eq), methyl 2-azidoacetate (6.61 g, 57.41 mmol, 3 eq) in MeOH (10 mL) was added NaOMe (4.36 M, 4.39 mL, 1 eq). The mixture was stirred at 0° C. for 2 hr. The reaction was monitored by LCMS, after the reaction was finished, the salt was removed by filtration and the filtrate was concentrated under reduced pressure to give the crude methyl (E)-2-azido-3-(4-bromo-5-fluoro-2-thienyl)prop-2-enoate (5 g, 16.33 mmol, 85.36% yield) as a yellow solid which was used directly in the next step without further purification.

### Step 3: methyl 3-bromo-2-fluoro-4H-thieno[3,2-b]pyrrole-5-carboxylate

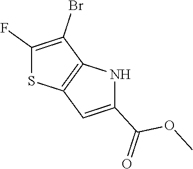

A solution of methyl (E)-2-azido-3-(4-bromo-5-fluoro-2-thienyl)prop-2-enoate (1.2 g, 39.2 mmol) in xylene (10 mL) was stirred at 140° C. for 3 hr. The reaction was monitored by TLC and LCMS, after the reaction was finished, the solvent was removed by concentration, the residue was purified by prep-HPLC (TFA condition) to give methyl 3-bromo-2-fluoro-4H-thieno[3,2-b]pyrrole-5-carboxylate (400 mg, 1.44 mmol, 36.69% yield) as a white solid.

### Step 4: methyl 3-bromo-2-fluoro-4-(4-(trifluoromethyl)benzyl)-4H-thieno[3,2-b]pyrrole-5-carboxylate

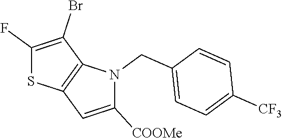 [SMILES: COC(=O)c1cc2sc(F)c(Br)c2n1Cc1ccc(C(F)(F)F)cc1]

To a solution of methyl 3-bromo-2-fluoro-4H-thieno[3,2-b]pyrrole-5-carboxylate (400 mg, 1.44 mmol, 1 eq), 1-(bromomethyl)-4-(trifluoromethyl)benzene (343.81 mg, 1.44 mmol, 221.81 μL, 1 eq) in DMF (10 mL) was added Cs₂CO₃ (1.41 g, 4.32 mmol, 3 eq). The mixture was stirred at room temperature for 2 hr. The reaction was monitored by TLC, after the reaction was finished, diluted with water and extracted with EtOAc (30 mL×2). The combined organic layers were washed with brine, dried over Na₂SO₄, filtered and concentrated under reduced pressure to give methyl 3-bromo-2-fluoro-4-[[4-(trifluoromethyl)phenyl]methyl]thieno[3,2-b]pyrrole-5-carboxylate (600 mg, 1.38 mmol, 95.63% yield) as a brown solid which was used directly in the next step without further purification.

### Step 5: 3-bromo-2-fluoro-4-(4-(trifluoromethyl)benzyl)-4H-thieno[3,2-b]pyrrole-5-carboxylic acid

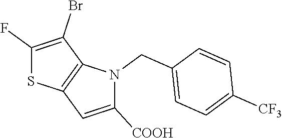 [SMILES: O=C(O)c1cc2sc(F)c(Br)c2n1Cc1ccc(C(F)(F)F)cc1]

To a solution of methyl 3-bromo-2-fluoro-4-[[4-(trifluoromethyl)phenyl]methyl]thieno[3,2-b]pyrr-ole-5-carboxylate (600 mg, 1.38 mmol, 1 eq) in MeOH (4 mL), THF (4 mL), H₂O (2 mL) was added LiOH (329.42 mg, 13.75 mmol, 10 eq). The mixture was stirred at 25° C. for 16 hr. The reaction was monitored by TLC, after the reaction was finished, diluted with water, and extracted with EtOAc (15 mL×2). The combined organic layers were washed with brine, dried over Na₂SO₄, filtered and concentrated under reduced pressure to give 3-bromo-2-fluoro-4-[[4-(trifluoromethyl)phenyl]methyl]thieno[3,2-b]pyrrole-5-carboxylic acid (200 mg, 473.72 μmol, 34.44% yield) as a white solid.

### Step 6: 3-bromo-2-fluoro-4-(4-(trifluoromethyl)benzyl)-4H-thieno[3,2-b]pyrrole

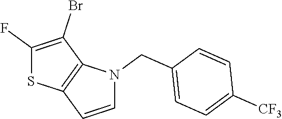 [SMILES: Fc1sc2ccn(Cc3ccc(C(F)(F)F)cc3)c2c1Br]

To a solution of 3-bromo-2-methyl-4-[[4-(trifluoromethyl)phenyl]methyl]thieno [3,2-b]pyrrole-5-carboxylic acid (50 mg, 119.55 μmol, 1 eq) in DMSO (1.5 mL) was added Ag₂CO₃ (32.97 mg, 119.55 μmol, 5.42 μL, 1 eq) and CH₃COOH (717.93 μg, 11.96 μmol, 0.1 eq). Then the mixture was stirred at 120° C. for 16 hr. The reaction was monitored by LCMS, after the reaction was finished, diluted with water, and extracted with EtOAc (15 mL×2). The combined organic layers were washed with brine, dried over Na₂SO₄, filtered and concentrated under reduced pressure to give 3-bromo-2-methyl-4-[[4-(trifluoromethyl)phenyl]methyl]thieno[3,2-b]pyrrole (20 mg, 53.44 μmol, 44.70% yield) as a white solid which was used directly in the next step without further purification.

### Step 7: 2-fluoro-4-(4-(trifluoromethyl)benzyl)-4H-thieno[3,2-b]pyrrole-3-carboxylic acid

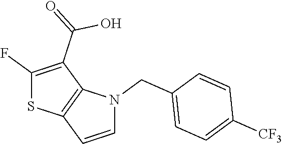 [SMILES: O=C(O)c1c(F)sc2ccn(Cc3ccc(C(F)(F)F)cc3)c12]

To a mixture of 3-bromo-2-fluoro-4-[[4-(trifluoromethyl)phenyl]methyl]thieno[3,2-b]pyrrole (140 mg, 370.19 μmol, 1 eq) in THF (3 mL) was added i-PrMgCl·LiCl (1.3 M, 370.19 μL, 1.3 eq) at −78° C. under N₂, then the reaction mixture was stirred at −10° C. for 20 min, then cooled back to −78° C. and bubbled with dry CO₂ for 20 min at −78° C. The reaction was monitored by LCMS, after the reaction was finished, the reaction was quenched by the addition of water and then extracted with EtOAc (10 mL×3). The organic layer was dried over Na₂SO₄, filtered and concentrated in vacuum to give 2-fluoro-4-[[4-(trifluoromethyl)phenyl]methyl]thieno[3,2-b]pyrrole-3-carboxylic acid (123 mg, 358.29 μmol, 96.79% yield) as a light-yellow oil, which was used directly to next step without further purification.

### Step 8: methyl 4-(1-(2-fluoro-4-(4-(trifluoromethyl)benzyl)-4H-thieno[3,2-b]pyrrole-3-carboxamido)cyclopropyl)benzoate

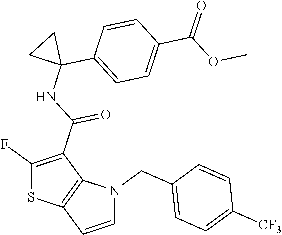 [SMILES: COC(=O)c1ccc(C2(NC(=O)c3c(F)sc4ccn(Cc5ccc(C(F)(F)F)cc5)c34)CC2)cc1]

To a solution of 2-fluoro-4-[[4-(trifluoromethyl)phenyl]methyl]thieno[3,2-b]pyrrole-3-carboxylic acid (120 mg, 349.55 μmol, 1 eq) in DMF (2 mL) was added HATU (172.78 mg, 454.42 μmol, 1.3 eq) and TEA (70.74 mg, 699.11 μmol, 97.31 μL, 2 eq) for 30 min and methyl 4-(1-aminocyclopropyl)benzoate (73.53 mg, 384.51 μmol, 1.1 eq) was added. The mixture was stirred at 25° C. for 12.5 hr. The reaction was monitored by LCMS, after the reaction was finished, the reaction mixture was concentrated under reduced pressure. Then diluted with water and extracted with EtOAc (15 mL×2). The combined organic layers were washed with brine, dried over Na₂SO₄, filtered, and concentrated under reduced pressure. The residue was purified by column chromatography (SiO₂, Petroleum ether/Ethyl acetate=3/1) to give methyl 4-[1-[[2-fluoro-4-[[4-(trifluoromethyl)phenyl]methyl]thieno[3,2-b]pyrrole-3-carbonyl]amino]cyclopropyl]benzoate (40 mg, 77.44 μmol, 22.15% yield) as a white solid.

MS (ES-API positive): 517.3 (M+1)⁺.

### Step 9: 4-(1-(2-fluoro-4-(4-(trifluoromethyl)benzyl)-4H-thieno[3,2-b]pyrrole-3-carboxamido)cyclopropyl)benzoic acid

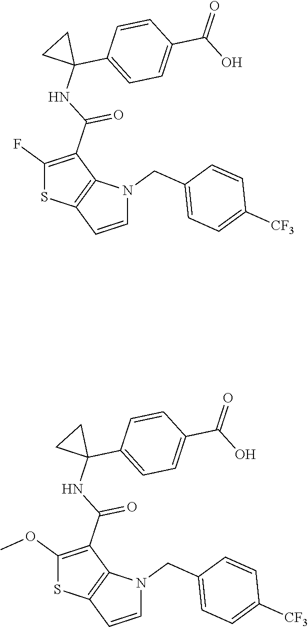 [SMILES: O=C(O)c1ccc(C2(NC(=O)c3c(F)sc4ccn(Cc5ccc(C(F)(F)F)cc5)c34)CC2)cc1 ; COc1sc2ccn(Cc3ccc(C(F)(F)F)cc3)c2c1C(=O)NC1(c2ccc(C(=O)O)cc2)CC1]

To a solution of methyl 4-[1-[[2-fluoro-4-[[4-(trifluoromethyl)phenyl]methyl thieno[3,2-b]pyrrole-3-carbonyl]amino]cyclopropyl]benzoate (30 mg, 58.08 μmol, 1 eq) in THF (1 mL), H₂O (0.3 mL) was added LiOH (13.91 mg, 580.83 μmol, 10 eq). The mixture was stirred at 40° C. for 12 hr. The reaction was monitored by LCMS. After the reaction was finished, the solvent was removed by concentration, the residue was purified by prep-HPLC (TFA condition). Column: Boston Green ODS 150×30 mm×5 μm; mobile phase: [water(TFA)-can]; B %: 53%-73%, 10 min followed by lyophilization to give 4-[1-[[2-fluoro-4-[[4-(trifluoromethyl)phenyl]methyl]thieno[3,2-b]pyrrole-3-carbonyl]amino]cyclopropyl]benzoic acid (20 mg, 39.80 μmol, 68.53% yield) as a white solid and methyl 4-[1-[[2-hydroxy-4-[[4-(trifluoromethyl)phenyl]methyl]thieno[3,2-b]pyrrole-3-carbonyl]amino]cyclopropyl]benzoate (5 mg, 9.72 μmol, 50.2% yield).

¹H NMR (500 MHz, DMSO) δ 12.78 (s, 1H), 9.20 (s, 1H), 7.77-7.75 (d, J=8.5 Hz, 2H), 7.62-7.61 (d, J=8.0 Hz, 2H), 7.23 (s, 1H), 7.17-7.16 (d, J=8.5 Hz, 2H), 7.07-7.05 (d, J=8.0 Hz, 2H), 6.48 (s, 1H), 5.56 (s, 2H), 1.26-1.24 (m, 2H), 1.03-0.97 (m, 2H); MS (ES-API positive): 503.1 (M+1)⁺.

¹H NMR (500 MHz, CD₃Cl) δ 7.93-7.91 (d, J=8.0 Hz, 2H), 7.47-7.45 (d, J=8.0 Hz, 2H), 7.15-7.13 (d, J=8.0 Hz, 2H), 7.03-7.02 (d, J=8.0 Hz, 2H), 6.37 (s, 1H), 5.82 (s, 2H), 4.08 (s, 3H), 1.46-1.44 (m, 2H), 1.29-1.24 (m, 2H).

MS (ES-API positive): 515.1 (M+1)⁺.

### Example 5: 3-[1-[[2-chloro-4-[[4-(trifluoromethyl)phenyl]methyl]thieno[3,2-b]pyrrole-3-carbonyl]amino]cyclopropyl]bicyclo[1.1.1]pentane-1-carboxylic acid

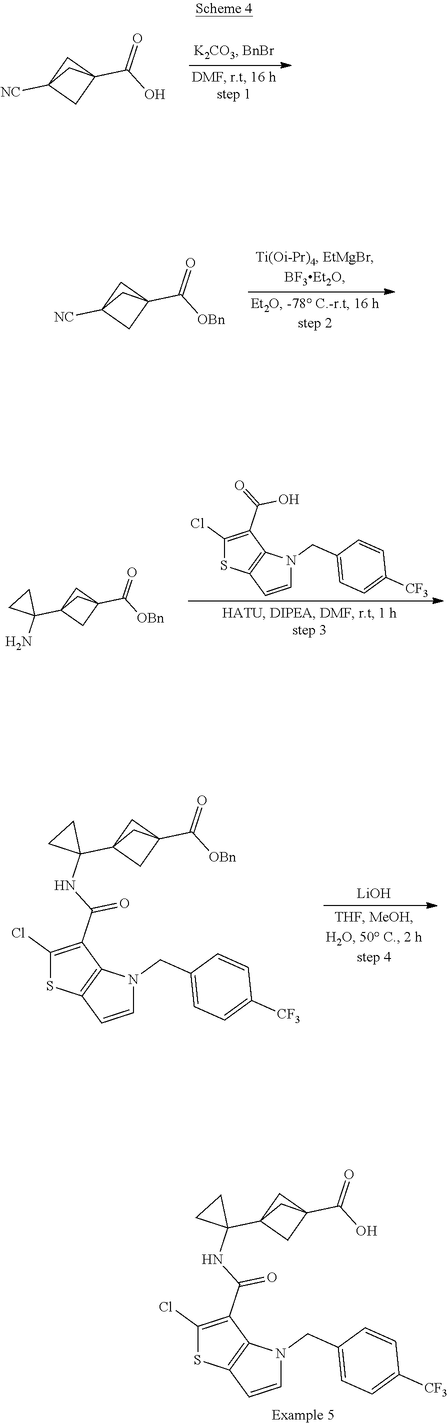

### Step 1: benzyl 3-cyanobicyclo[1.1.1]pentane-1-carboxylate

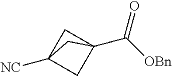 [SMILES: N#CC12CC(C(=O)OCc3ccccc3)(C1)C2]

To a solution of 3-cyanobicyclo[1.1.1]pentane-1-carboxylic acid (0.5 g, 3.65 mmol) in DMF (5 mL) was added K₂CO₃ (1.01 g, 7.29 mmol) and benzyl bromide (748.31 mg, 4.38 mmol, 519.66 μL). The mixture was stirred at room temperature for 16 hr. The reaction was monitored by TLC, after the reaction was finished, the reaction mixture was diluted with water and extracted with EtOAc (10 mL×3). The combined organic layers were washed with brine, dried over Na₂SO₄, filtered, benzyl bromide and concentrated under reduced pressure; the residue was purified via silica gel column (EtOAc in Petroleum ether gradient from 0 to 5%) to give 390 mg of the title product as a colorless oil.

MS (ES-API positive): 227.1 (M+1)⁺.

### Step 2: benzyl 3-(1-aminocyclopropyl)bicyclo[1.1.1]pentane-1-carboxylate

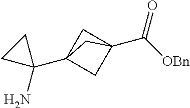 [SMILES: NC1(C23CC(C(=O)OCc4ccccc4)(C2)C3)CC1]

To a solution of benzyl 3-cyanobicyclo[1.1.1]pentane-1-carboxylate (390 mg, 1.72 mmol) in Et₂O (10 mL) under N₂ atmosphere was added Ti(Oi-Pr)₄ (536.52 mg, 1.89 mmol, 557.13 μL) at room temperature, then the reaction was cooled to −78° C. and EtMgBr (3 M, 1.26 mL) was added at −78° C. dropwise, after the addition was finished, the reaction was stirred at −78° C. for 1 h, then BF₃·Et₂O (487.13 mg, 3.43 mmol, 423.59 μL) was added at −78° C. dropwise, then the reaction was slowly warm to room temperature (25° C.) and stirred at 25° C. for 16 hr. under N₂ atmosphere. The reaction was monitored by LCMS, after the addition was finished, the reaction mixture was quenched by 1 M HCl dropwise at 25° C., diluted with H₂O, then 10% aqueous NaOH (15 mL) was added dropwise at 25° C. until the PH=7, after stirred at room temperature for 20 min, then extracted with EtOAc (20 mL×2). The combined organic layers were dried over Na₂SO₄, concentrated under reduced pressure to give 300 mg of the title product as a yellow oil which was used directly in the next step.

MS (ES-API positive): 258.3 (M+1)⁺.

### Step 3: benzyl 3-[1-[[2-chloro-4-[[4-(trifluoromethyl)phenyl]methyl]thieno[3,2-b]pyrrole-3-carbonyl]amino]cyclopropyl]bicyclo[1.1.1]pentane-1-carboxylate

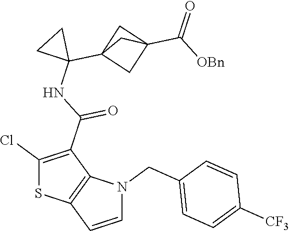 [SMILES: O=C(NC1(C23CC(C(=O)OCc4ccccc4)(C2)C3)CC1)c1c(Cl)sc2ccn(Cc3ccc(C(F)(F)F)cc3)c12]

To a solution of benzyl 3-(1-aminocyclopropyl)bicyclo[1.1.1]pentane-1-carboxylate (160.94 mg, 625.43 μmol) and 2-chloro-4-[[4-(trifluoromethyl)phenyl]methyl]thieno[3,2-b]pyrrole-3-carboxylic acid (150 mg, 416.96 μmol) in DMF (5 mL) was added HATU (475.62 mg, 1.25 mmol) and DIEA (80.83 mg, 625.43 μmol, 108.94 μL at 25° C. The mixture was stirred at 25° C. for 1 hr. The reaction was monitored by LCMS, after the reaction was finished, the reaction mixture was diluted with water and extracted with EtOAc (15 mL×3). The combined organic layers were washed with brined then concentrated under reduced pressure to give 200 mg of the title product as a brown oil which was used directly in the next step without further purification.

MS (ES-API positive): 598.1 (M+1)⁺.

### Step 4: 3-[1-[[2-chloro-4-[[4-(trifluoromethyl)phenyl]methyl]thieno[3,2-b]pyrrole-3-carbonyl]amino]cyclopropyl]bicyclo[1.1.1]pentane-1-carboxylic acid

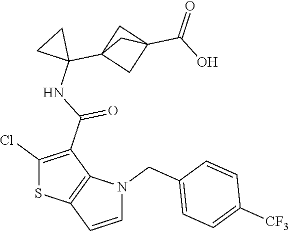 [SMILES: O=C(NC1(C23CC(C(=O)O)(C2)C3)CC1)c1c(Cl)sc2ccn(Cc3ccc(C(F)(F)F)cc3)c12]

To a solution of benzyl 3-[1-[[2-chloro-4-[[4-(trifluoromethyl)phenyl]methyl]thieno[3,2-b]pyrrole-3-carbonyl]amino]cyclopropyl]bicyclo[1.1.1]pentane-1-carboxylate (200 mg, 333.86 μmol) in MeOH (5 mL), THF (5 mL) and H₂O (2.5 mL) was added LiOH (79.95 mg, 3.34 mmol. The mixture was stirred at 55° C. for 2 hr. The reaction was monitored by LCMS, after the reaction was finished, the reaction mixture was quenched by 1M HCl until PH=4-5, then extracted with EtOAc (15 mL, 3 times). The combined organic layers were concentrated under reduced pressure, the residue was purified via prep-HPLC (HCl condition: column: Boston Green ODS 150×30 mm×5 μm; mobile phase: [water(HCl)-ACN]; B %: 52%-72%, 10 min) to give 34.46 mg of the title product as a white solid.

¹H NMR (CD₃OD, 400 MHz) δ 8.79 (s, 1H), 7.58 (d, 2H, J=8.2 Hz), 7.10 (d, 1H, J=3.0 Hz), 7.05 (br d, 2H, J=8.1 Hz), 6.45 (d, 1H, J=3.0 Hz), 5.53 (s, 2H), 1.82 (s, 6H), 0.70-0.60 (m, 2H), 0.40-0.30 (m, 2H).

MS (ES-API positive): 509.0 (M+1)⁺.

### Example 6. 4-[1-[[2-chloro-4-[[4-(trifluoromethyl)phenyl]methyl]thieno[3,2-b]pyrrole-3-carbonyl]amino]cyclopropyl]bicyclo[2.2.2]octane-1-carboxylic acid

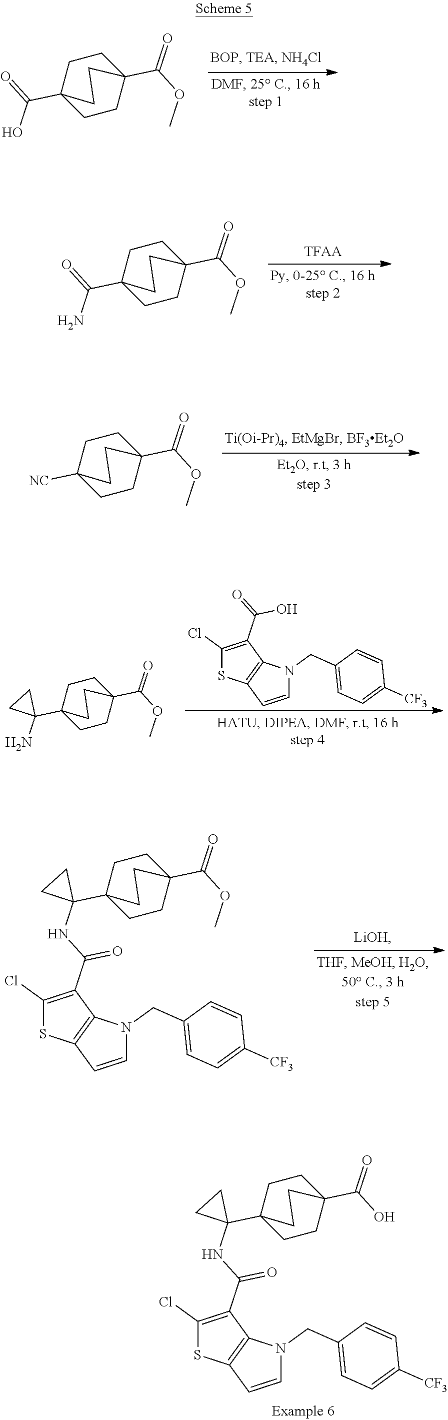

### Step 1: methyl 4-carbamoylbicyclo [2.2.2]octane-1-carboxylate

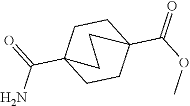

A mixture of 4-methoxycarbonylbicyclo [2.2.2]octane-1-carboxylic acid (10 g, 47.12 mmol), BOP (20.84 g, 47.12 mmol), TEA (19.07 g, 188.46 mmol, 26.23 mL) and NH₄Cl (25.20 g, 471.16 mmol) in DMF (100 mL) was stirred at 25° C. for 16 hr. The reaction was monitored by LCMS, after the reaction was finished, the reaction mixture was diluted with water (˜50 mL) and extracted with EtOAc (50 mL×3). The combined organic layers were washed with brine and dried over Na₂SO₄, filtered and concentrated under reduced pressure to give 9.9 g of the crude title compound as a pale-yellow oil which was used directly in the next step.

MS (ES-API positive): 212.3 (M+1)⁺.

### Step 2: methyl 4-cyanobicyclo [2.2.2]octane-1-carboxylate

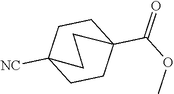

To a solution of methyl 4-carbamoylbicyclo [2.2.2]octane-1-carboxylate (9.9 g, 46.86 mmol) in Pyridine (80 mL) was added TFAA (68.90 g, 328.04 mmol, 45.63 mL) dropwise at 0° C. Then the mixture was slowly warmed to room temperature e and stirred overnight. The reaction was monitored by TLC, after the reaction was finished, the reaction mixture was diluted with 1N HCl and extracted with EtOAc (30 mL×3). The combined organic layers were washed with brine and dried over Na₂SO₄, filtered and concentrated under reduced pressure, the residue was purified via silica gel column (EtOAc in Petroleum ether gradient from 0 to 20%) to give 9 g of the title product as a yellow solid.

MS (ES-API positive): 193.1 (M+1)⁺.

### Step 3: methyl 4-(1-aminocyclopropyl)bicyclo[2.2.2]octane-1-carboxylate

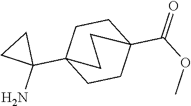 [SMILES: COC(=O)C12CCC(C3(N)CC3)(CC1)CC2]

To a solution of methyl 4-cyanobicyclo[2.2.2]octane-1-carboxylate (470 mg, 2.43 mmol) in Et₂O (30 mL) under N₂ atmosphere was added Ti(Oi-Pr)₄ (760.38 mg, 2.68 mmol, 789.59 μL) at 25° C., then the reaction was cooled to −78° C. and EtMgBr (3 M, 1.78 mL) was added at −78° C. dropwise, after the addition was finished, the reaction was stirred at −78° C. for 1 hr., then BF₃·Et₂O (690.40 mg, 4.86 mmol, 600.35 μL) was added at −78° C. dropwise, then the reaction was slowly warm to room temperature and stirred at 25° C. for 5 hr. under N₂ atmosphere. The reaction was monitored by LCMS, after the reaction was finished, the reaction was quenched by 1 M HCl dropwise at room temperature and then diluted with water, then 10% aqueous NaOH (15 mL) was added dropwise at 25° C. until the PH=7, after stirred at room temperature for 20 min, extracted with EtOAc (20 mL×2). The combined organic layers were dried over Na₂SO₄, concentrated under reduced pressure to give 350 mg of the title product as a yellow oil which was used in the next step without purification.

MS (ES-API positive): 224.3 (M+1)⁺.

### Step 4: methyl 4-[1-[[2-chloro-4-[[4-(trifluoromethyl)phenyl]methyl]thieno[3,2-b]pyrrole-3-carbonyl]amino]cyclopropyl]bicyclo[2.2.2]octane-1-carboxylate

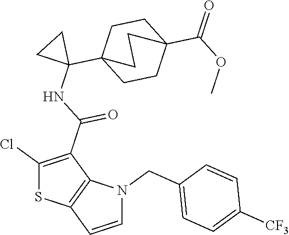 [SMILES: COC(=O)C12CCC(C3(NC(=O)c4c(Cl)sc5ccn(Cc6ccc(C(F)(F)F)cc6)c45)CC3)(CC1)CC2]

To a solution of 2-chloro-4-[[4-(trifluoromethyl)phenyl]methyl]thieno[3,2-b]pyrrole-3-carboxylic acid (100 mg, 277.97 μmol) in DMF (5 mL) was added and methyl 4-(1-aminocyclopropyl)bicyclo[2.2.2]octane-1-carboxylate (124.15 mg, 277.97 μmol, 50% purity), DIEA (107.77 mg, 833.91 μmol, 145.25 μL) and HATU (158.54 mg, 416.96 μmol). The mixture was stirred at 25° C. for 16 hr, the reaction was monitored by LCMS, after the reaction was finished, the reaction mixture was diluted with H₂O and extracted with EtOAc (15 mL×3). The combined organic layers were washed with brine, then concentrated under reduced pressure to give 150 mg of the title product as a brown oil which was used directly in the next step without purification.

MS (ES-API positive): 565.1 (M+1)⁺.

### Step 5: 4-[1-[[2-chloro-4-[[4-(trifluoromethyl)phenyl]methyl]thieno[3,2-b]pyrrole-3-carbonyl]amino]cyclopropyl]bicyclo[2.2.2]octane-1-carboxylic acid

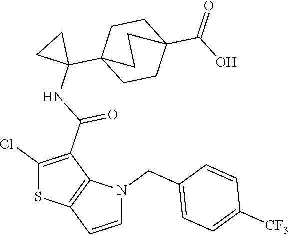 [SMILES: O=C(NC1(C23CCC(C(=O)O)(CC2)CC3)CC1)c1c(Cl)sc2ccn(Cc3ccc(C(F)(F)F)cc3)c12]

To a solution of methyl 4-[1-[[2-chloro-4-[[4-(trifluoromethyl)phenyl]methyl]thieno[3,2-b]pyrrole-3-carbonyl]amino]cyclopropyl]bicyclo[2.2.2]octane-1-carboxylate (150 mg, 265.47 μmol) in THF (5 mL), MeOH (5 mL) and H2O (2.5 mL) was added LiOH (63.58 mg, 2.65 mmol). The mixture was stirred at 55° C. for 3 hr, the reaction was monitored by LCMS, after the reaction was finished, the reaction mixture was diluted with H₂O and 1M HCl until PH=4-5, then extracted with EtOAc (15 mL×3). The combined organic layers were concentrated under reduced pressure, the residue was purified via prep-HPLC (HCl condition column: Boston Green ODS 150×30 mm×5 μm; mobile phase: [water (HCl)-ACN]; B %: 70%-90%, 10 min) to give 45.48 mg of the title product as a white solid.

¹H NMR (CD₃OD, 400 MHz) δ 8.62 (s, 1H), 7.56 (d, 2H, J=8.0 Hz), 7.13-7.02 (m, 3H), 6.42 (d, 1H, J=3.2 Hz), 5.50 (s, 2H), 1.65-1.61 (m, 6H), 1.42-1.38 (m, 6H), 0.73-0.70 (m, 2H), 0.28-0.25 (m, 2H).

MS (ES-API positive): 551.1 (M+1)⁺.

### Example 7: 3-[1-[[2-methyl-4-[[4-(trifluoromethyl)phenyl]methyl]thieno[3,2-b]pyrrole-3-carbonyl]amino]cyclopropyl]bicyclo[1.1.1]pentane-1-carboxylic acid

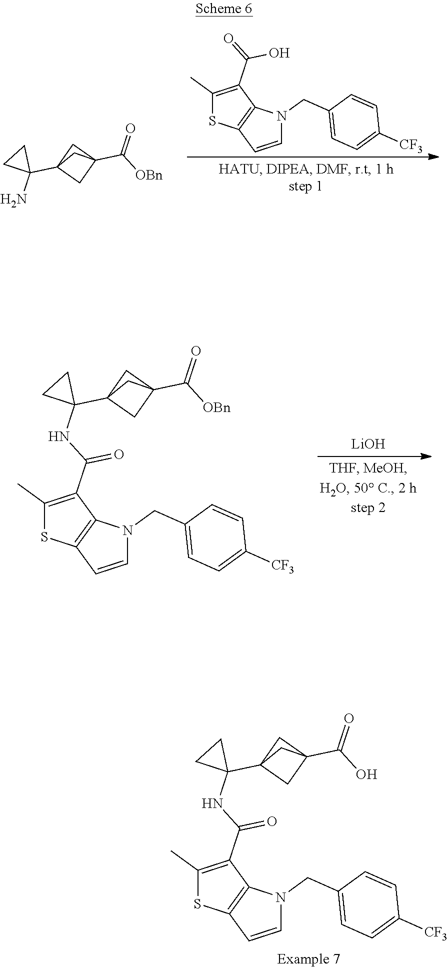 [SMILES: NC1(C23CC(C(=O)OCc4ccccc4)(C2)C3)CC1 ; Cc1sc2ccn(Cc3ccc(C(F)(F)F)cc3)c2c1C(=O)O ; Cc1sc2ccn(Cc3ccc(C(F)(F)F)cc3)c2c1C(=O)NC1(C23CC(C(=O)OCc4ccccc4)(C2)C3)CC1 ; "LiOH" ; Cc1sc2ccn(Cc3ccc(C(F)(F)F)cc3)c2c1C(=O)NC1(C23CC(C(=O)O)(C2)C3)CC1]

### Step 1: benzyl 3-[1-[[2-methyl-4-[[4-(trifluoromethyl)phenyl]methyl]thieno[3,2-b]pyrrole-3-carbonyl]amino]cyclopropyl]bicyclo[1.1.1]pentane-1-carboxylate

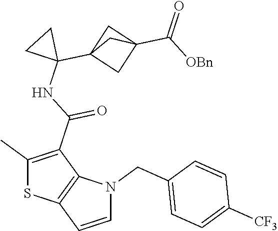 [SMILES: Cc1sc2ccn(Cc3ccc(C(F)(F)F)cc3)c2c1C(=O)NC1(C23CC(C(=O)OCc4ccccc4)(C2)C3)CC1]

To a solution of benzyl 3-(1-aminocyclopropyl)bicyclo[1.1.1]pentane-1-carboxylate (170.63 mg, 663.07 μmol) and 2-methyl-4-[[4-(trifluoromethyl)phenyl]methyl]thieno[3,2-b]pyrrole-3-carboxylic acid (150 mg, 442.05 μmol) in DMF (5 mL) was added HATU (252.12 mg, 663.07 μmol) and DIEA (171.39 mg, 1.33 mmol, 230.98 μL) at 25° C. The mixture was stirred at 25° C. for 1 hr. The reaction was monitored by LCMS, after the reaction was finished, the reaction mixture was quenched with water and extracted with EtOAc (15 mL×3). The combined organic layers were washed with brine, then concentrated under reduced pressure to give 200 mg of the title product as a brown oil which was used directly in the next step without further purification.

MS (ES-API positive): 579.3 (M+1)⁺.

### Step 2: 3-[1-[[2-methyl-4-[[4-(trifluoromethyl)phenyl]methyl]thieno[3,2-b]pyrrole-3-carbonyl]amino]cyclopropyl]bicyclo[1.1.1]pentane-1-carboxylic acid

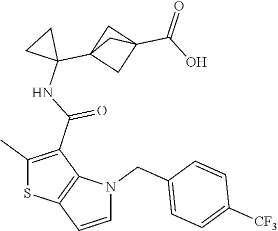 [SMILES: Cc1sc2ccn(Cc3ccc(C(F)(F)F)cc3)c2c1C(=O)NC1(C23CC(C(=O)O)(C2)C3)CC1]

To a solution of benzyl 3-[1-[[2-methyl-4-[[4-(trifluoromethyl)phenyl]methyl]thieno[3,2-b]pyrrole-3-carbonyl]amino]cyclopropyl]bicyclo[1.1.1]pentane-1-carboxylate (0.2 g, 345.64 μmol) in MeOH (5 mL), THF (5 mL) and H₂O (2.5 mL) was added LiOH (82.78 mg, 3.46 mmol). The mixture was stirred at 50° C. for 2 hr. The reaction was monitored by LCMS, after the reaction was finished, the reaction mixture was diluted with water, then 1M HCl was added until pH=4-5, then extracted with EtOAc (15 mL×3). The combined organic layers were concentrated under reduced pressure, the residue was purified via prep-HPLC ((HCl condition: column: Boston Green ODS 150×30 mm×5 μm; mobile phase: [water (HCl)-ACN]; B %: 50%-70%, 10 min) to give 55.14 mg of the title product as a white solid.

¹H NMR (CD₃OD, 400 MHz) δ 8.58 (s, 1H), 7.53 (d, 2H, J=8.0 Hz), 7.13-6.92 (m, 3H), 6.38 (d, 1H, J=3.2 Hz), 5.51 (s, 2H), 2.57 (s, 3H), 1.77 (s, 6H), 0.71-0.63 (m, 2H), 0.42-0.33 (m, 2H).

MS (ES-API positive): 489.0 (M+1)⁺.

### Example 8: 4-[1-[[2-chloro-4-[[4-(trifluoromethyl)phenyl]methyl]thieno[3,2-b]pyrrole-3-carbonyl]amino]cyclopropyl]bicyclo[2.2.2]octane-1-carboxylic acid

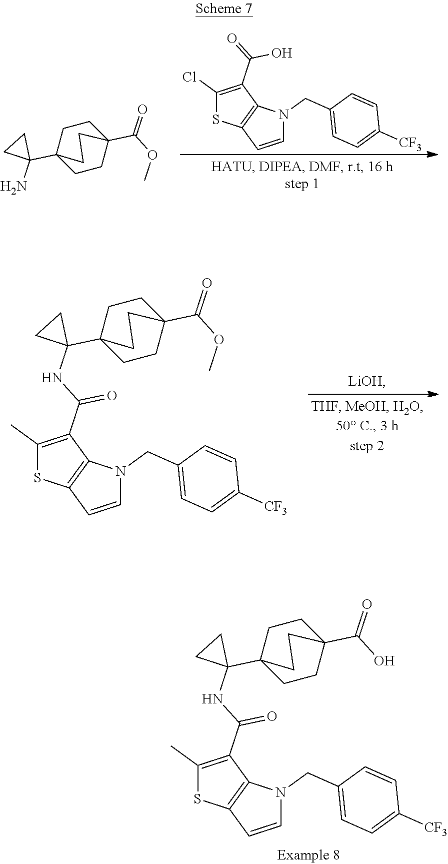

### Step 1: methyl 4-[1-[[2-methyl-4-[[4-(trifluoromethyl)phenyl]methyl]thieno[3,2-b]pyrrole-3-carbonyl]amino]cyclopropyl]bicyclo[2.2.2]octane-1-carboxylate

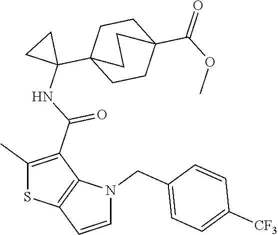 [SMILES: COC(=O)C12CCC(C3(NC(=O)c4c(C)sc5ccn(Cc6ccc(C(F)(F)F)cc6)c45)CC3)(CC1)CC2]

To a solution of 2-methyl-4-[[4-(trifluoromethyl)phenyl]methyl]thieno[3,2-b]pyrrole-3-carboxylic acid (150 mg, 442.05 μmol) in DMF (5 mL) was added methyl 4-(1-aminocyclopropyl)bicyclo[2.2.2]octane-1-carboxylate (296.14 mg, 663.07 μmol, 50% purity) DIEA (171.39 mg, 1.33 mmol, 230.98 μL) and HATU (252.12 mg, 663.07 μmol). The mixture was stirred at 25° C. for 16 hr, the reaction was monitored by LCMS, after the reaction was finished, the reaction was quenched with water and extracted with EtOAc (15 mL×3). The combined organic layers were washed with brine, then concentrated under reduced pressure to give 0.2 g of the title compound as a brown oil which was used directly in the next step without further purification.

MS (ES-API positive): 545.4 (M+1)⁺.

### Step 2: 4-[1-[[2-methyl-4-[[4-(trifluoromethyl)phenyl]methyl]thieno[3,2-b]pyrrole-3-carbonyl]amino]cyclopropyl]bicyclo[2.2.2]octane-1-carboxylic acid

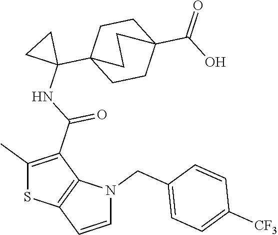 [SMILES: Cc1sc2ccn(Cc3ccc(C(F)(F)F)cc3)c2c1C(=O)NC1(C23CCC(C(=O)O)(CC2)CC3)CC1]

To a solution of methyl 4-[1-[[2-methyl-4-[[4-(trifluoromethyl)phenyl]methyl]thieno[3,2-b]pyrrole-3-carbonyl]amino]cyclopropyl]bicyclo[2.2.2]octane-1-carboxylate (0.2 g, 367.22 μmol) in THF (5 mL), MeOH (5 mL) and H₂O (2.5 mL) was added LiOH (380 mg, 15.87 mmol). The mixture was stirred at 50° C. for 3 hr. The reaction was monitored by LCMS, after the reaction was finished, the reaction mixture was diluted with H₂O (10 mL), then 1M HCl was added until PH=4-5, then extracted with EtOAc (15 mL×3). The combined organic layers were concentrated under reduced pressure. The residue was purified via prep-HPLC (HCl condition; column: Boston Green ODS 150×30 mm×5 μm; mobile phase: [water (HCl)-ACN]; B %: 56%-76%, 10 min) to give 14.59 mg of the title product as a white solid.

¹H NMR (CD₃OD, 400 MHz) δ 8.47 (s, 1H), 7.57 (d, 2H, J=8.0 Hz), 7.12-6.93 (m, 3H), 6.39 (d, 1H, J=3.2 Hz), 5.53 (s, 2H), 2.57 (s, 3H), 1.72-1.64 (m, 6H), 1.43-1.36 (m, 6H), 0.82-0.75 (m, 2H), 0.33-0.28 (m, 2H).

MS (ES-API positive): 531.0 (M+1)⁺.

### Example 9: 3-[1-[[2-chloro-4-[[4-(trifluoromethyl)phenyl]methyl]thieno[3,2-b]pyrrole-3-carbonyl]amino]ethyl]bicyclo[1.1.1]pentane-1-carboxylic acid

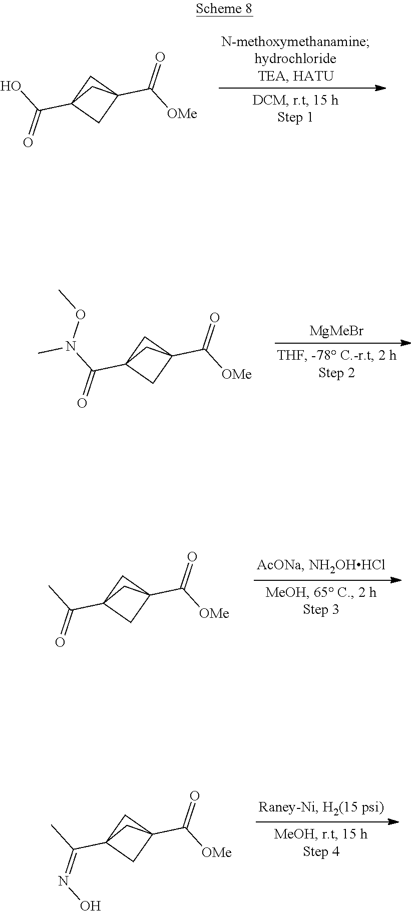

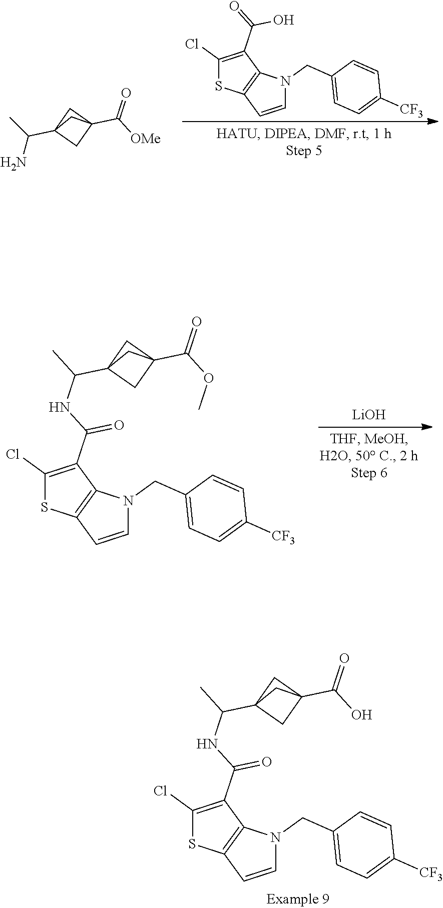 [SMILES: COC(=O)C12CC(C(C)N)(C1)C2 ; O=C(O)c1c(Cl)sc2ccn(Cc3ccc(C(F)(F)F)cc3)c12 ; COC(=O)C12CC(C(C)NC(=O)c3c(Cl)sc4ccn(Cc5ccc(C(F)(F)F)cc5)c34)(C1)C2 ; "LiOH" ; CC(NC(=O)c1c(Cl)sc2ccn(Cc3ccc(C(F)(F)F)cc3)c12)C12CC(C(=O)O)(C1)C2]

### Step 1: methyl 3-[methoxy(methyl)carbamoyl]bicyclo[1.1.1]pentane-1-carboxylate

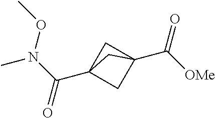

To a solution of 3-methoxycarbonylbicyclo[1.1.1]pentane-1-carboxylic acid (1.0 g, 5.88 mmol, 1 eq), N-methoxymethanamine; hydrochloride (687.89 mg, 7.05 mmol, 1.2 eq) and Et₃N (1.78 g, 17.63 mmol, 2.45 mL, 3.0 eq) in DCM (15 mL) was added HATU (2.68 g, 7.05 mmol, 1.2 eq). The mixture was stirred at room temperature overnight. The reaction was monitored by LCMS, after the reaction was finished, the reaction was quenched with water, and then extracted with DCM (10 mL×3). The combined organic layers were concentrated under reduced pressure. The residue was purified by flash silica gel chromatography (ISCO®; 12 g SepaFlash® Silica Flash Column, Eluent of 0˜25% Ethyl acetate/Petroleum ether gradient @30 mL/min) give 950 mg of the title product as a white solid. MS (ES-API positive): 214.2 (M+1)⁺.

### Step 2: methyl 3-acetylbicyclo[1.1.1]pentane-1-carboxylate

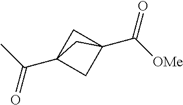

To a solution of methyl 3-[methoxy(methyl)carbamoyl]bicyclo[1.1.1]pentane-1-carboxylate (200 mg, 937.96 μmol, 1 eq) in THF (10 mL) was added MeMgBr (3 M, 468.98 μL, 1.5 eq) at −78° C., after the addition was finished, the reaction was slowly warm to room temperature and stirred at room temperature for 2 hr. The reaction was monitored by TLC, after the reaction was finished, the reaction mixture was quenched by the addition of saturated NH₄Cl, and then diluted with water and extracted with ethyl acetate (10 mL×3). The combined organic layers were concentrated under reduced pressure. The residue was purified by flash silica gel chromatography (ISCO®; 4 g SepaFlash® Silica Flash Column, Eluent of 0˜20% Ethyl acetate/Petroleum ether gradient @30 mL/min) to give 70 mg of the title product as a white solid.

### Step 3: methyl 3-[(Z)—N-hydroxy-C-methyl-carbonimidoyl]bicyclo[1.1.1]pentane-1-carboxylate

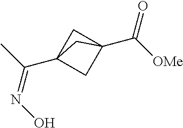

A mixture of methyl 3-acetylbicyclo[1.1.1]pentane-1-carboxylate (70 mg, 416.20 μmol, 1 eq), AcONa (102.43 mg, 1.25 mmol, 3 eq) and hydroxylammonium Chloride (86.77 mg, 1.25 mmol, 3 eq) in MeOH (5 mL) was stirred at 65° C. for 2 hr. under N₂ atmosphere. The reaction was monitored by LCMS, after the reaction was finished, the reaction mixture was concentrated under reduced pressure to remove MeOH. The residue was diluted with ethyl acetate, washed with brine (10 mL×2). The organic layers were concentrated under reduced pressure to give 70 mg of the title product as a pale white solid which was used directly in the next step without further purification.

MS (ES-API positive): 184.1 (M+1)+

### Step 4: methyl 3-(1-aminoethyl)bicyclo[1.1.1]pentane-1-carboxylate

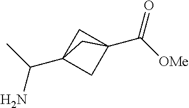

A mixture of methyl 3-[(Z)—N-hydroxy-C-methyl-carbonimidoyl]bicyclo[1.1.1]pentane-1-carboxylate (70 mg, 382.09 μmol, 1 eq), Raney-Ni (51.07 mg, 596.06 μmol, 1.56 eq) in MeOH (3 mL) was degassed and purged with H₂ for 3 times, and then the mixture was stirred at 25° C. for 15 hr under H₂ atmosphere. The reaction was monitored by LCMS, after the reaction was finished, the catalyst was removed by filtration and the filtrate was concentrated under reduced pressure to 60 mg of the title product as a pale colorless oil which was used directly in the next step without further purification.

MS (ES-API positive): 170.2 (M+1)⁺.

### Step 5: methyl 3-[1-[[2-chloro-4-[[4-(trifluoromethyl)phenyl]methyl]thieno[3,2-b]pyrrole-3-carbonyl]amino]ethyl]bicyclo[1.1.1]pentane-1-carboxylate

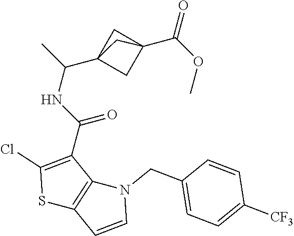 [SMILES: COC(=O)C12CC(C(C)NC(=O)c3c(Cl)sc4ccn(Cc5ccc(C(F)(F)F)cc5)c34)(C1)C2]

To a solution of methyl 3-(1-aminoethyl)bicyclo[1.1.1]pentane-1-carboxylate (60 mg, 354.57 μmol, 1 eq) in DMF (5 mL) was added 2-chloro-4-[[4-(trifluoromethyl)phenyl]methyl]thieno[3,2-b]pyrrole-3-carboxylic acid (127.56 mg, 354.57 μmol, 1 eq) HATU (161.78 mg, 425.48 μmol, 1.2 eq) and DIPEA (137.48 mg, 1.06 mmol, 185.28 μL, 3 eq). The mixture was stirred at room temperature for 1 hr. The reaction was monitored by LCMS, after the reaction was finished, the reaction mixture was quenched with water and extracted with ethyl acetate (3×10 mL). The combined organic layers were concentrated under reduced pressure to give 150 mg of the title product as a brown oil which was used directly in the next step without further purification.

MS (ES-API positive): 511.2 (M+1)⁺.

### Step 6: 3-[1-[[2-chloro-4-[[4-(trifluoromethyl)phenyl]methyl]thieno[3,2-b]pyrrole-3-carbonyl]amino]ethyl]bicyclo[1.1.1]pentane-1-carboxylic acid

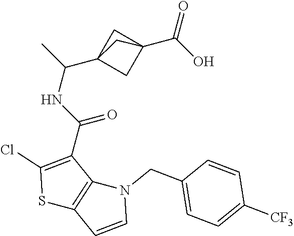 [SMILES: CC(NC(=O)c1c(Cl)sc2ccn(Cc3ccc(C(F)(F)F)cc3)c12)C12CC(C(=O)O)(C1)C2]

To a solution of methyl 3-[1-[[2-chloro-4-[[4-(trifluoromethyl)phenyl]methyl]thieno[3,2-b]pyrrole-3-carbonyl]amino]ethyl]bicyclo[1.1.1]pentane-1-carboxylate (150 mg, 293.57 μmol, 1 eq) in THF (1 mL), MeOH (1 mL) and H₂O (0.5 mL) was added LiOH (70.30 mg, 2.94 mmol, 10 eq). The mixture was stirred at 50° C. for 2 hr. The reaction was monitored by LCMS. After the reaction was completed, the reaction mixture was concentrated under reduced pressure to remove solvent, and then diluted with water and adjusted to pH=4˜5 with 1N HCl (aq), and extracted with ethyl acetate (3×10 mL). The combined organic layers were washed with brine, and then concentrated under reduced pressure. The residue was purified by reversed-phase HPLC (column: Boston Green ODS 150×30 mm×5 μm; mobile phase: [water (HCl)-ACN]; B %: 52%-72%, 10 min) to give 61.2 mg of the title product as a pale white solid.

¹H NMR (400 MHz, CD₃OD) δ 8.32 (br d, J=8.0 Hz, 1H), 7.56 (d, J=8.4 Hz, 2H), 7.14-7.10 (m, 3H), 6.43 (d, J=2.8 Hz, 1H), 5.58-5.40 (m, 2H), 4.02-3.95 (m, 1H), 1.94-1.84 (m, 6H), 0.83 (d, J=6.8 Hz, 3H).

MS (ES-API positive): 497.0 (M+1)⁺.

### Example 10: 3-[1-[[2-methyl-4-[[4-(trifluoromethyl)phenyl]methyl]thieno[3,2-b]pyrrole-3-carbonyl]amino]ethyl]bicyclo[1.1.1]pentane-1-carboxylic acid

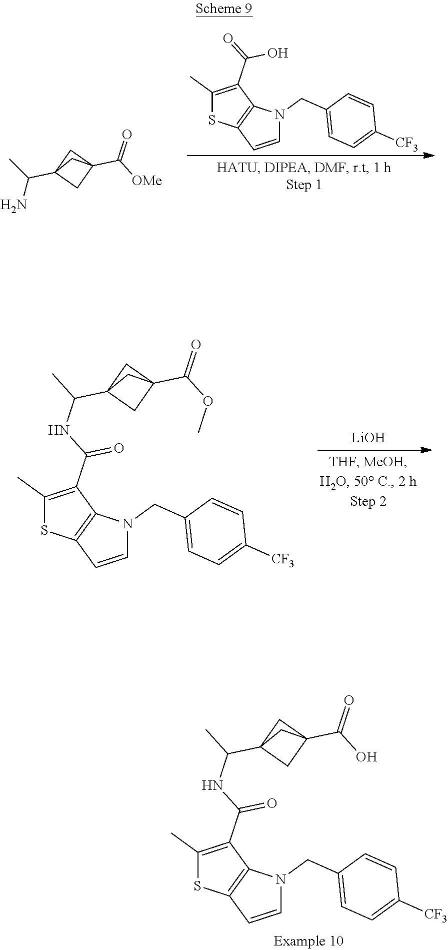 [SMILES: COC(=O)C12CC(C(C)N)(C1)C2 ; Cc1sc2ccn(Cc3ccc(C(F)(F)F)cc3)c2c1C(=O)O ; COC(=O)C12CC(C(C)NC(=O)c3c(C)sc4ccn(Cc5ccc(C(F)(F)F)cc5)c34)(C1)C2 ; "LiOH" ; Cc1sc2ccn(Cc3ccc(C(F)(F)F)cc3)c2c1C(=O)NC(C)C12CC(C(=O)O)(C1)C2]

### Step 1: methyl 3-[1-[[2-methyl-4-[[4-(trifluoromethyl)phenyl]methyl]thieno[3,2-b]pyrrole-3-carbonyl]amino]ethyl]bicyclo[1.1.1]pentane-1-carboxylate

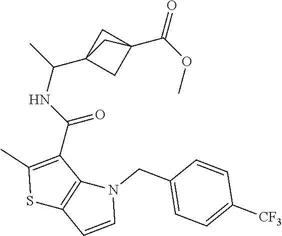 [SMILES: COC(=O)C12CC(C(C)NC(=O)c3c(C)sc4ccn(Cc5ccc(C(F)(F)F)cc5)c34)(C1)C2]

To a solution of methyl 3-(1-aminoethyl)bicyclo[1.1.1]pentane-1-carboxylate (50 mg, 295.47 μmol, 1 eq) and 2-methyl-4-[[4-(trifluoromethyl)phenyl]methyl]thieno[3,2-b]pyrrole-3-carboxylic acid (100.26 mg, 295.47 μmol, 1 eq) in DMF (3 mL) was added HATU (134.82 mg, 354.56 μmol, 1.2 eq) and DIPEA (114.56 mg, 886.41 μmol, 154.40 μL, 3 eq). The mixture was stirred at room temperature for 1 hr. The reaction was monitored by LCMS, after the reaction was finished, the reaction was quenched with water and extracted with ethyl acetate (3×10 mL). The combined organic layers were concentrated under reduced pressure to give 140 mg of the title product as a white solid which was used directly in the next step without further purification.

MS (ES-API positive): 491.2 (M+1)⁺.

### Step 2: 3-[1-[[2-methyl-4-[[4-(trifluoromethyl)phenyl]methyl]thieno[3,2-b]pyrrole-3-carbonyl]amino]ethyl]bicyclo[1.1.1]pentane-1-carboxylic acid

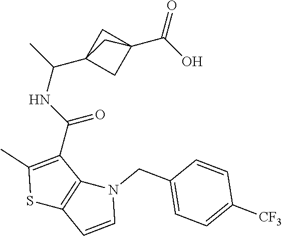 [SMILES: Cc1sc2ccn(Cc3ccc(C(F)(F)F)cc3)c2c1C(=O)NC(C)C12CC(C(=O)O)(C1)C2]

To a solution of methyl 3-[1-[[2-methyl-4-[[4-(trifluoromethyl)phenyl]methyl]thieno[3,2-b]pyrrole-3-carbonyl]amino]ethyl]bicyclo[1.1.1]pentane-1-carboxylate (140 mg, 285.40 μmol, 1 eq) in THF (2 mL), MeOH (2 mL) and water (1 mL) was added LiOH (68.35 mg, 2.85 mmol, 10 eq). The mixture was stirred at 50° C. for 2 hr. The reaction was monitored by LCMS, after the reaction was finished, most of the solvent was removed by concentration, the residue was diluted with water and adjusted to pH=4˜5 with 1N HCl (aq), then extracted with ethyl acetate (10 mL×3). The combined organic layers were washed with brine and then concentrated under reduced pressure, the residue was purified by reversed-phase HPLC (column: Boston Green ODS 150×30 mm×5 μm; mobile phase: [water (HCl)-ACN]; B %: 50%-70%, 10 min) to give 47.7 mg of the title product as a pale white solid.

¹H NMR (400 MHz, CD₃OD) δ 8.11 (br d, J=8.0 Hz, 1H), 7.54 (d, J=8.0 Hz, 2H), 7.08 (d, J=8.0 Hz, 2H), 6.99 (d, J=3.2 Hz, 1H), 6.38 (d, J=3.2 Hz, 1H), 5.60-5.40 (m, 2H), 4.03-3.96 (m, 1H), 2.56 (s, 3H), 1.82 (s, 6H), 0.84 (d, J=6.8 Hz, 3H).

MS (ES-API positive): 477.1 (M+1)⁺.

### Example 11: 4-(1-(2-(trifluoromethyl)-4-(4-(trifluoromethyl)benzyl)-4H-thieno[3,2-b]pyrrole-3-carboxamido)cyclopropyl)benzoic acid

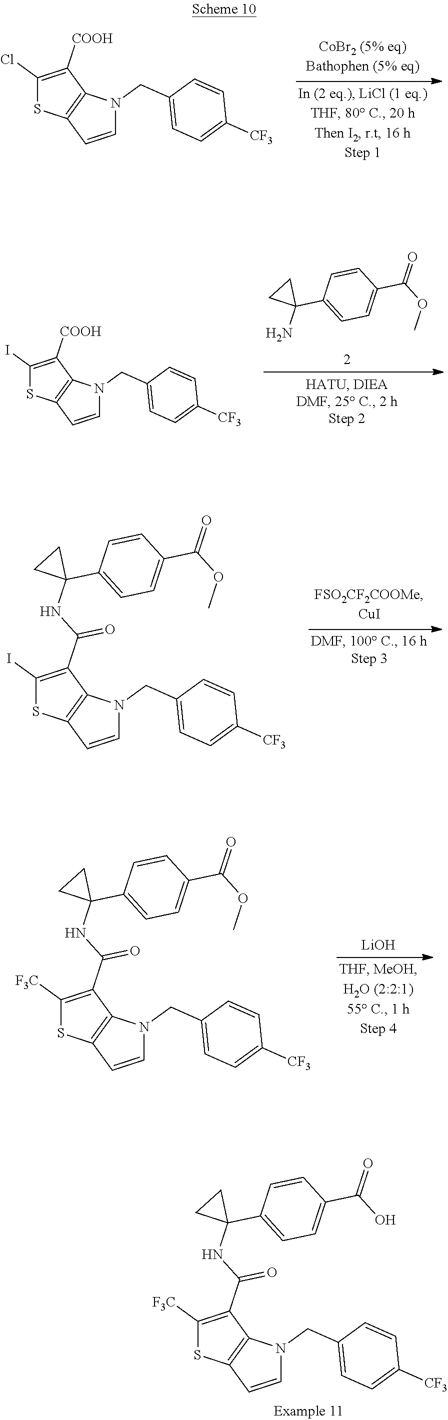

### Step 1: 2-iodo-4-(4-(trifluoromethyl)benzyl)-4H-thieno[3,2-b]pyrrole-3-carboxylic acid

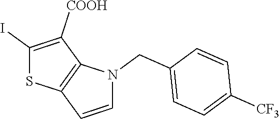 [SMILES: O=C(O)c1c(I)sc2ccn(Cc3ccc(C(F)(F)F)cc3)c12]

A mixture of 2-chloro-4-[[4-(trifluoromethyl)phenyl]methyl]thieno[3,2-b]pyrrole-3-carboxylic acid (0.2 g, 555.94 μmol, 1 eq), LiCl (47.14 mg, 1.11 mmol, 22.77 μL, 2 eq), TMSCl (2.42 mg, 22.24 μmol, 2.82 μL, 0.04 eq), dibromo cobalt (12.16 mg, 55.59 μmol, 0.1 eq), 4,7-diphenyl-1,10-phenanthroline (18.48 mg, 55.59 μmol, 0.1 eq), 167.98 μL, 1.5 eq) and indigane (255.33 mg, 2.22 mmol, 34.98 μL, 4 eq) in THF (5 mL) was stirred at 80° C. for 20 hr, then the reaction cooled to room temperature and molecular iodine (211.65 mg, 833.91 μmol) was added and the reaction was stirred at room temperature overnight, the reaction was monitored by LCMS, after the reaction was finished, the salt was removed by filtration and the filtrate was concentrated under reduced pressure. The residue was purified by prep-HPLC (FA condition: column: Boston Green ODS 150×30 mm×5 μm; mobile phase: [water(FA)-ACN]; B %: 62%-92%, 12 min) to give 2-iodo-4-[[4-(trifluoromethyl)phenyl]methyl]thieno[3,2-b]pyrrole-3-carboxylic acid (42 mg, 93.08 μmol, 16.74% yield) as a pale pink solid.

MS (ES-API positive): 451.9 (M+1)⁺.

### Step 2: methyl 4-(1-(2-iodo-4-(4-(trifluoromethyl)benzyl)-4H-thieno[3,2-b]pyrrole-3-carboxamido)cyclopropyl)benzoate

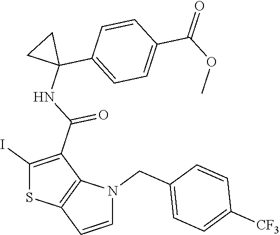 [SMILES: COC(=O)c1ccc(C2(NC(=O)c3c(I)sc4ccn(Cc5ccc(C(F)(F)F)cc5)c34)CC2)cc1]

To a solution of 2-iodo-4-[[4-(trifluoromethyl)phenyl]methyl]thieno[3,2-b]pyrrole-3-carboxylic acid (42 mg, 93.08 μmol, 1 eq) and methyl 4-(1-aminocyclopropyl)benzoate (26.70 mg, 139.63 μmol, 1.5 eq) in DMF (2 mL) was added HATU (106.18 mg, 279.25 μmol, 3 eq) and DIEA (18.05 mg, 139.63 μmol, 24.32 μL, 1.5 eq). The mixture was stirred at 25° C. for 2 hr. The reaction was monitored by LCMS, after the reaction was finished, the reaction was quenched by the addition of water and then extracted with ethyl acetate (10 mL×3), the organic layers were collected and concentrated under reduced pressure. The residue was purified by prep-HPLC (FA condition; column: Boston Green ODS 150×30 mm×5 μm; mobile phase: [water(FA)-ACN]; B %: 53%-83%, 12 min) to give methyl 4-[1-[[2-iodo-4-[[4-(trifluoromethyl)phenyl]methyl]thieno[3,2-b]pyrrole-3-carbonyl]amino]cyclopropyl]benzoate (26 mg, 41.64 μmol, 44.73% yield) as a white solid.

MS (ES-API positive): 625.0 (M+1)⁺.

### Step 3: methyl 4-(1-(2-(trifluoromethyl)-4-(4-(trifluoromethyl)benzyl)-4H-thieno[3,2-b]pyrrole-3-carboxamido)cyclopropyl)benzoate

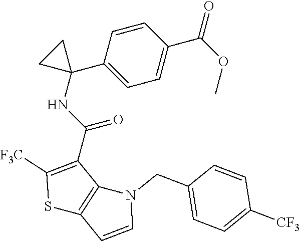 [SMILES: COC(=O)c1ccc(C2(NC(=O)c3c(C(F)(F)F)sc4ccn(Cc5ccc(C(F)(F)F)cc5)c34)CC2)cc1]

To a solution of methyl 4-[1-[[2-iodo-4-[[4-(trifluoromethyl)phenyl]methyl]thieno[3,2-b]pyrrole-3-carbonyl]amino]cyclopropyl]benzoate (16 mg, 25.62 μmol, 1 eq) in DMF (1.5 mL) was added CuI (14.64 mg, 76.87 μmol, 3 eq), methyl 2,2-difluoro-2-fluorosulfonyl-acetate (14.77 mg, 76.87 μmol, 9.78 μL, 3 eq). Then the mixture was stirred at 100° C. for 16 hr, the reaction was monitored by LCMS, after the reaction was finished, the salt was removed by filtration and the filtrated was concentrated under reduced pressure. The residue was purified by prep-HPLC (FA condition: column: Boston Green ODS 150×30 mm×5 um; mobile phase: [water (FA)-ACN]; B %: 65%-95%, 12 min) to give methyl 4-[1-[[2-(trifluoromethyl)-4-[[4-(trifluoromethyl)phenyl]methyl]thieno[3,2-b]pyrrole-3-carbonyl]amino]cyclopropyl]benzoate (8 mg, 14.12 μmol, 55.11% yield) as a white solid.

MS (ES-API positive): 567.2 (M+1)⁺.

### Step 4: 4-(1-(2-(trifluoromethyl)-4-(4-(trifluoromethyl)benzyl)-4H-thieno[3,2-b]pyrrole-3-carboxamido)cyclopropyl)benzoic acid

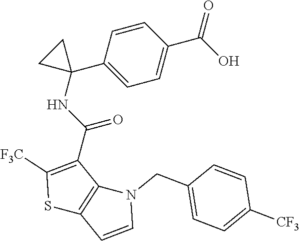 [SMILES: O=C(O)c1ccc(C2(NC(=O)c3c(C(F)(F)F)sc4ccn(Cc5ccc(C(F)(F)F)cc5)c34)CC2)cc1]

To a solution of methyl 4-[1-[[2-(trifluoromethyl)-4-[[4-(trifluoromethyl)phenyl]methyl]thieno[3,2-b]pyrrole-3-carbonyl]amino]cyclopropyl]benzoate (11 mg, 19.42 μmol, 1 eq) in MeOH (1 mL), THF (1 mL) and H₂O (0.5 mL) was added LiOH (4.65 mg, 194.17 μmol, 10 eq). The mixture was stirred at 55° C. for 1 hr. The reaction was monitored by LCMS, after the reaction was finished, the reaction was quenched by the addition of 1 M HCl until PH=3˜4, then diluted with water and extracted with EtOAc (3×10 mL). The organic layers were collected and concentrated under reduced pressure. The residue was purified by prep-HPLC (HCl condition; column: Boston Green ODS 150×30 mm×5 μm; mobile phase: [water(HCl)-ACN]; B %: 56%-76%, 10 min) to give 4-[1-[[2-(trifluoromethyl)-4-[[4-(trifluoromethyl)phenyl]methyl]thieno[3,2-b]pyrrole-3-carbonyl]amino]cyclopropyl]benzoic acid (3 mg, 5.43 μmol, 27.97% yield) as a white solid.

¹H NMR (400 MHz, CD₃OD) δ 9.48 (s, 1H), 7.67 (d, J=8.4 Hz, 2H), 7.43 (d, J=8.4 Hz, 2H), 7.26 (d, J=8.4 Hz, 2H), 7.03 (d, J=2.8 Hz, 1H), 6.88 (d, J=8.4 Hz, 2H), 6.46 (d, J=2.8 Hz, 1H), 5.16 (s, 2H), 1.15-1.12 (m, 2H), 1.01-0.98 (m, 2H).

MS (ES-API positive): 553.2 (M+1)⁺.

The examples and illustrations above are not intended to limit the scope of this invention. Any combination of embodiments of this invention, along with any obvious extension or analogs, are within the scope of this invention. Further, it is intended that this invention encompass any arrangement, which is calculated to achieve that same purpose, and all such variations and modifications are fall within the scope of the appended claims.

1. A compound of Formula I 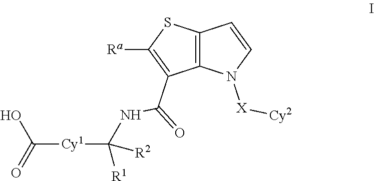
wherein:
R¹ and R² are each independently hydrogen, C₁₋₆ alkyl, C₁₋₆ cycloalkyl, C₁₋₆ halo-cycloalkyl, or C₁₋₆ haloalkyl; or, R¹ and R², together with the carbon atom to which they are both attached, form a 3- to 6-membered carbocyclic ring which is optionally substituted with one to three R^{a} groups and optionally contains one or two ring-forming heteroatom(s) each independently being S, O, or NR^{b}, wherein each R^{b} is independently hydrogen, C₁₋₆ alkyl, C₁₋₆ cycloalkyl, C₁₋₆ halo-cycloalkyl, and C₁₋₆ haloalkyl, aryl, heteroaryl, —C(O)—C₁₋₆alkyl, —C(O)-aryl, —S(O)₂-alkyl, or —S(O)₂-aryl;
X is absent, ═CH—, —CR¹R²—, or —C(O)—;
Cy¹ is C₁₋₆ alkylene, C₁₋₆ alkenylene, C₁₋₆ alkynylene, cycloalkylene, cycloalkenylene arylene, heteroarylene, heterocyclylene, or bridged bicyclic cycloalkylene or cycloalkenylene, and each of C₁₋₆ alkylene, C₁₋₆ alkenylene, C₁₋₆ alkynylene, cycloalkylene, cycloalkenylene, arylene, heteroarylene, heterocyclylene, or bridged bicyclic cycloalkylene is optionally substituted;
Cy² is cycloalkyl, aryl, heteroaryl, or heterocyclyl, and is optionally substituted with one to three substitution groups each independently being halo, alkyl or haloalkyl; and
each R^{a} group is independently halo, alkyl, haloalkyl, hydroxyalkyl, or alkoxy; and
when R^{a} is alkyl, Cy¹ is bridged bicyclic cycloalkylene;
or a pharmaceutically acceptable salt thereof.

2. The compound of claim 1, wherein X is —CH₂—.

3. The compound of claim 1, wherein Cy² is aryl and optionally substituted with one haloalkyl.

4. The compound of claim 1, wherein halo is —F or —Cl.

5. The compound of claim 1, wherein Cy¹ is arylene or bridged bicyclic cycloalkylene.

6. The compound of claim 1, wherein R¹ and R² are each independently hydrogen or C₁₋₆ alkyl; or, R¹ and R², together with the carbon atom to which they are both attached, form a 3- to 6-membered carbocyclic ring.

7. The compound of claim 1, wherein R^{a} is —F, —Cl, —CF₃, hydroxyalkyl, alkoxyl or —CH₃; and when R^{a} is —CH₃, Cy¹ is C₅-C₁₀ bridged bicyclic cycloalkylene.

8. The compound of claim 1, wherein the compound is of Formula II, 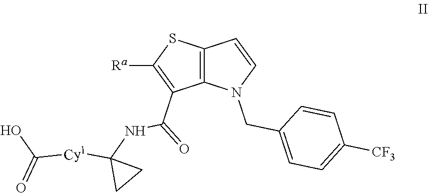
wherein Cy¹ is arylene or bridged bicyclic cycloalkylene; and R^{a} is halo, alkyl, haloalkyl, hydroxyalkyl, or alkoxy; when R^{a} is alkyl, Cy¹ is bridged bicyclic cycloalkylene.

9. The compound of claim 8, wherein Cy¹ is phenylene or C₅-C₁₀ bridged bicyclic cycloalkylene.

10. The compound of claim 9, wherein the C₅-C₁₀ bridged bicyclic cycloalkylene is

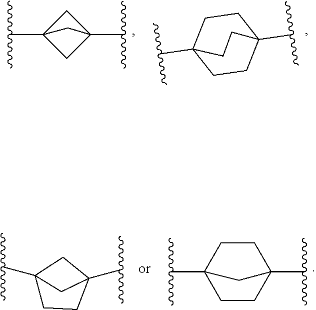

11. The compound of claim 1, wherein the compound is

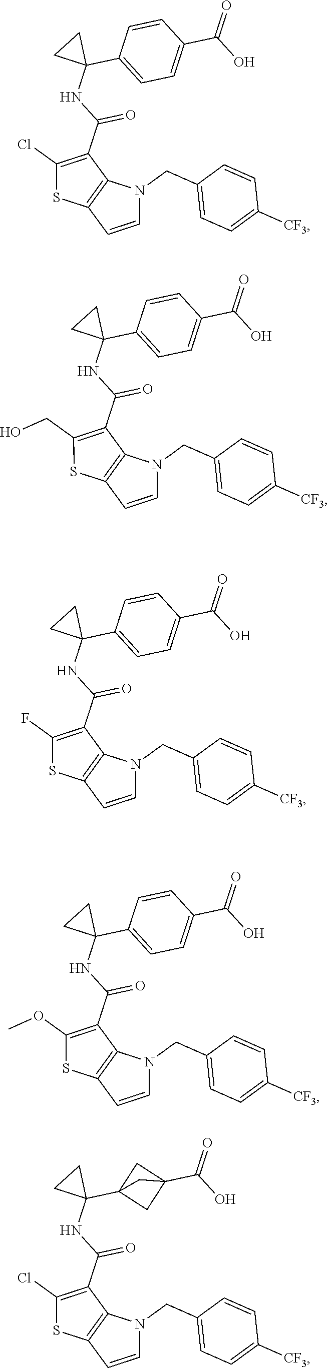 [SMILES: O=C(O)c1ccc(C2(NC(=O)c3c(Cl)sc4ccn(Cc5ccc(C(F)(F)F)cc5)c34)CC2)cc1 ; O=C(O)c1ccc(C2(NC(=O)c3c(CO)sc4ccn(Cc5ccc(C(F)(F)F)cc5)c34)CC2)cc1 ; O=C(O)c1ccc(C2(NC(=O)c3c(F)sc4ccn(Cc5ccc(C(F)(F)F)cc5)c34)CC2)cc1 ; COc1sc2ccn(Cc3ccc(C(F)(F)F)cc3)c2c1C(=O)NC1(c2ccc(C(=O)O)cc2)CC1 ; O=C(NC1(C23CC(C(=O)O)(C2)C3)CC1)c1c(Cl)sc2ccn(Cc3ccc(C(F)(F)F)cc3)c12]

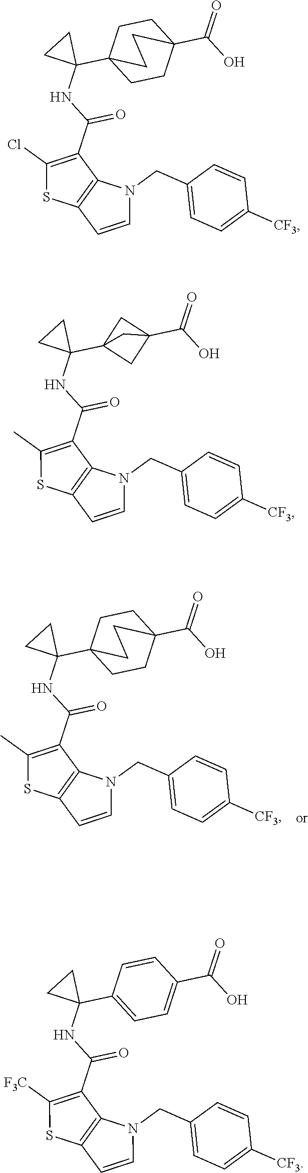 [SMILES: O=C(NC1(C23CCC(C(=O)O)(CC2)CC3)CC1)c1c(Cl)sc2ccn(Cc3ccc(C(F)(F)F)cc3)c12 ; Cc1sc2ccn(Cc3ccc(C(F)(F)F)cc3)c2c1C(=O)NC1(C23CC(C(=O)O)(C2)C3)CC1 ; Cc1sc2ccn(Cc3ccc(C(F)(F)F)cc3)c2c1C(=O)NC1(C23CCC(C(=O)O)(CC2)CC3)CC1 ; O=C(O)c1ccc(C2(NC(=O)c3c(C(F)(F)F)sc4ccn(Cc5ccc(C(F)(F)F)cc5)c34)CC2)cc1]

12. A pharmaceutical composition comprising a compound of claim 1 or a pharmaceutically acceptable salt thereof and a pharmaceutically acceptable carrier or excipient.

13. The pharmaceutical composition of claim 12, further comprising a therapeutic agent selected from the group consisting of antibodies to cytotoxic t-lymphocyte antigen 4 (anti-CTLA4), antibodies to programmed death ligand 1 (anti-PD L1), antibodies to programmed cell death protein 1 (anti-PD1), indoleamine-2,3-dioxygenase (IDO) inhibitors, and tryptophan-2,3-dioxygenase (TDO) inhibitors and antimetabolites.

14. The pharmaceutical composition of claim 12, wherein the composition is used in combination with a radiation therapy agent.

15. A method for treating a subject suffering from a condition mediated by the action of PGE2 at EP4 receptors, comprising administering to the subject in need thereof an effective amount of a compound of claim 1.

16. The method of claim 15, wherein the condition is an inflammatory disease or cancer.

17. The method of claim 16, wherein the inflammatory disease is arthritis, acne vulgaris, asthma, autoimmune diseases, autoinflammatory diseases, Celiac disease, chronic prostatitis, colitis, diverticulitis, glomerulonephritis, hidradenitis suppurativa, hypersensitivities, inflammatory bowel diseases, interstitial cystitis, Mast Cell Activation Syndrome, macrocytosis, otitis, pelvic inflammatory disease, reperfusion injury, rheumatic fever, rheumatoid arthritis, rhinitis, sarcoidosis, or vasculitis.

18. The method of claim 16, wherein the cancer is breast cancer, endometrial cancer, cervix cancer, ovary cancer, lung cancer, head and neck cancer, brain cancer, thyroid cancer, esophagus cancer, stomach cancer, colon and rectal cancer, liver cancer, pancreatic cancer, skin cancer, kidney cancer, bladder cancer, prostate cancer, testis cancer, bone cancer, Lymphoma, or blood cancer.

19. (canceled)

20. (canceled)
